(19) United States
(12) Patent Application Publication
(10) Pub. No.: US 2017/0066727 A1
(43) Pub. Date: Mar. 9, 2017

(54) Photostabilizing Compounds and Methods of Making and Using the Same

(71) Applicant: Elevance Renewable Sciences, Inc., Woodbridge, IL (US)

(72) Inventors: Ryan Littich, Powell, OH (US); Georgeta Hategan, Plainfield, IL (US)

(73) Assignee: Elevance Renewable Sciences, Inc., Woodridge, IL (US)

(21) Appl. No.: 15/183,555

(22) Filed: Jun. 15, 2016

Related U.S. Application Data
(60) Provisional application No. 62/186,481, filed on Jun. 30, 2015.

(51) Int. Cl. C07D 249/20; A61K 8/06; A61Q 17/04; A61K 8/49

(52) U.S. Cl. CPC C07D 249/20; A61K 8/496; A61K 8/06; A61Q 17/04

(57) ABSTRACT

Compounds that stabilize ultraviolet (UV) light are generally disclosed herein. Methods of making and using such compounds are also generally disclosed. Compositions, such as sunscreen compositions, containing such UV-stabilizing compounds are also generally disclosed herein. In some embodiments, UV-stabilizing compounds disclosed herein are derived from a natural oil, such as by metathesis.

### CROSS-REFERENCE TO RELATED APPLICATIONS

The present application claims the benefit of priority of U.S. Provisional Application No. 62/186,481, filed Jun. 30, 2015, which is incorporated herein by reference as though set forth herein in its entirety.

### TECHNICAL FIELD

Compounds that stabilize ultraviolet (UV) light are generally disclosed herein. Methods of making and using such compounds are also generally disclosed. Compositions, such as sunscreen compositions, containing such photostabilizing compounds are also generally disclosed herein. In some embodiments, photostabilizing compounds disclosed herein are derived from a natural oil, such as by metathesis.

### BACKGROUND

Sunscreen compositions may contain a variety of ingredients for filtering various wavelengths of UV light. Some of these ingredients function as physical UV filters, and function primarily by scattering UV light. Common examples include zinc oxide and titanium dioxide. These ingredients are typically photostable, but may not protect against all relevant wavelengths of UV light, and can also make the sunscreen composition appear white and pasty. Therefore, such ingredients are often used in combination with chemical UV filters, which function by absorbing UV light at the relevant wavelengths. Avobenzone is one of the more common such compounds used in sunscreen compositions. Such chemical UV filters are often not photostable, meaning that they have a tendency to break down into compounds that offer little UV filtering capability.

Such a lack of photostability can be illustrated with avobenzone. In its ground state, avobenzone exists in a singlet electronic state. When it absorbs UV radiation, certain electrons in the compound are excited to a singlet excited state. Desirably, the excited-state compound dissipates the absorbed energy through a nonradiative process and returns to the ground-state singlet state, where it can continue to function as a UV filter. In some instances, however, the excited-state compound can undergo intersystem crossing to a triplet excited state (e.g., a diradical state). When this occurs, it is unlikely that the compound will be able to return to its original singlet ground state, as such transitions are electronically forbidden. Thus, the diradical triplet ends up decaying through some other path, such as by hemolysis. In most cases, such processes result in compounds that function as poor UV filters, thereby diminishing the UV-filtering capability of the applied composition.

Photostabilizers are compounds that can prolong the useful life of chemical UV filters by increasing the likelihood that the excited-state compound transitions back to its original singlet ground state instead of undergoing intersystem crossing and undergoing hemolysis. Therefore, photostabilizers are commonly used in sunscreen compositions. Useful photostabilizers include certain small-molecule compounds and polymers. These each have different advantages. Thus, there is a continuing need for new photostabilizing compounds that can provide a broader range of use.

### SUMMARY

In a first aspect, the disclosure provides compounds of formula (I):

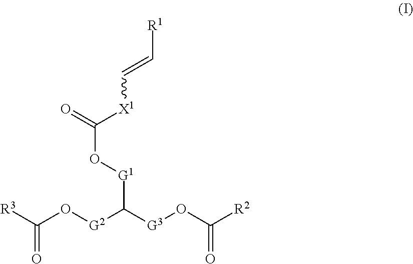

wherein: X¹ is C₁₋₂₀ alkylene, C₂₋₂₀ alkenylene, C₁₋₂₀ heteroalkylene, or C₂₋₂₀ heteroalkenylene, each of which is optionally substituted one or more times by R^{x}; two of G¹, G², and G³ are —CH₂—, and one of G¹, G², and G³ is a direct bond; R¹ is a moiety comprising a UV-stabilizing residue, such as —X⁴—R⁸, wherein X⁴ is C₁₋₂₀ hydrocarbylene, which is optionally substituted one or more times by R^{x}, and R⁸ is a UV-stabilizing residue; R² is C₁₋₂₄ alkyl, C₂₋₂₄ alkenyl, C₁₋₂₄ heteroalkyl, or C₂₋₂₄ heteroalkenyl, each of which is optionally substituted one or more times by R^{x}, or R² is a moiety of the following formula:

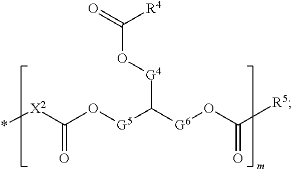

R³ is C₁₋₂₄ alkyl, C₂₋₂₄ alkenyl, C₁₋₂₄ heteroalkyl, or C₂₋₂₄ heteroalkenyl, each of which is optionally substituted one or more times by R^{x}, or R³ is a moiety of the following formula:

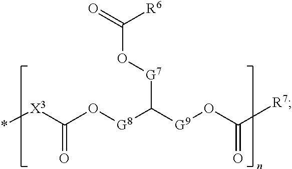

X² and X³ are independently C₁₋₃₀ alkylene, C₂₋₃₀ alkenylene, C₁₋₃₀ heteroalkylene, or C₂₋₃₀ heteroalkenylene, each of which is optionally substituted one or more times by R^{x}; two of G⁴, G⁵, and G⁶ are —CH₂—, and one of G⁴, G⁵, and G⁶ is a direct bond, wherein, if m is greater than 1, these assignments are made independently for each repeating unit; two of G⁷, G⁸, and G⁹ are —CH₂—, and one of G⁷, G⁸, and G⁹ is a direct bond, wherein, if n is greater than 1, these assignments are made independently for each repeating unit; R⁴ and R⁶ are independently C₁₋₂₄ alkyl, C₂₋₂₄ alkenyl, C₁₋₂₄ heteroalkyl, or C₂₋₂₄ heteroalkenyl, each of which is optionally substituted one or more times by R^{x}, or are independently a moiety comprising a UV-stabilizing residue, such as —X¹⁴—R¹⁸, wherein X¹⁴ is C₁₋₂₀ hydrocarbylene, which is optionally substituted one or more times by R^{x}, and R¹⁸ is a UV-stabilizing residue; R⁵ and R⁷ are independently C₁₋₂₄ alkyl, C₂₋₂₄ alkenyl, C₁₋₂₄ heteroalkyl, or C₂₋₂₄ heteroalkenyl, each of which is optionally substituted one or more times by R^{x}; m and n are independently 0, 1, 2, 3, 4, or 5; and R^{x} is a halogen atom, —OH, —NH₂, C₁₋₆ alkyl, C₁₋₆ heteroalkyl, C₂₋₆ alkenyl, or C₂₋₆ heteroalkenyl.

In a second aspect, the disclosure provides compounds formed by reacting one or more unsaturated glycerides with a compound of formula (II)

HC═CH—R²¹ (II)

in the presence of a metathesis catalyst, wherein R²¹ is a moiety comprising a UV-stabilizing residue.

In a third aspect, the disclosure provides methods for making a photostabilizing composition, the method comprising: providing (a) one or more unsaturated glycerides, and (b) one or more olefinically functionalized UV-stabilizing compounds; and reacting the one or more unsaturated glycerides and the one or more olefinically functionalized UV-stabilizing compounds in the presence of a metathesis catalyst.

In a fourth aspect, the disclosure provides compositions comprising compounds of the first and/or second aspects or any embodiments thereof. In some embodiments, the compositions are personal care compositions. In some embodiments, the compositions are sunscreen compositions.

Further aspects and embodiments are provided in the foregoing drawings, detailed description and claims.

### BRIEF DESCRIPTION OF THE DRAWINGS

The following drawings are provided for purposes of illustrating various embodiments of the compositions and methods disclosed herein. The drawings are provided for illustrative purposes only, and are not intended to describe any preferred compositions or preferred methods, or to serve as a source of any limitations on the scope of the claimed inventions.

FIG. 1 shows the UV-Visible absorption spectrum for compositions disclosed herein and a comparative compound.

FIG. 2 illustrates the UV stabilization of avobenzone for a comparative composition.

FIG. 3 illustrates the UV stabilization of avobenzone for a composition disclosed herein.

### DETAILED DESCRIPTION

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

### Definitions

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

As used herein, “polymer” refers to a substance having a chemical structure that includes the multiple repetition of constitutional units formed from substances of comparatively low relative molecular mass relative to the molecular mass of the polymer. The term “polymer” includes soluble and/or fusible molecules having chains of repeat units, and also includes insoluble and infusible networks. As used herein, the term “polymer” can include oligomeric materials, which have only a few (e.g., 5-100) constitutional units

As used herein, “natural oil,” “natural feedstock,” or “natural oil feedstock” refer to oils derived from plants or animal sources. These terms include natural oil derivatives, unless otherwise indicated. The terms also include modified plant or animal sources (e.g., genetically modified plant or animal sources), unless indicated otherwise. Examples of natural oils include, but are not limited to, vegetable oils, algae oils, fish oils, animal fats, tall oils, derivatives of these oils, combinations of any of these oils, and the like. Representative non-limiting examples of vegetable oils include rapeseed oil (canola oil), coconut oil, corn oil, cottonseed oil, olive oil, palm oil, peanut oil, safflower oil, sesame oil, soybean oil, sunflower oil, linseed oil, palm kernel oil, tung oil, jatropha oil, mustard seed oil, pennycress oil, camelina oil, hempseed oil, and castor oil. Representative non-limiting examples of animal fats include lard, tallow, poultry fat, yellow grease, and fish oil. Tall oils are by-products of wood pulp manufacture. In some embodiments, the natural oil or natural oil feedstock comprises one or more unsaturated glycerides (e.g., unsaturated triglycerides). In some such embodiments, the natural oil feedstock comprises at least 50% by weight, or at least 60% by weight, or at least 70% by weight, or at least 80% by weight, or at least 90% by weight, or at least 95% by weight, or at least 97% by weight, or at least 99% by weight of one or more unsaturated triglycerides, based on the total weight of the natural oil feedstock.

As used herein, “natural oil derivatives” refers to the compounds or mixtures of compounds derived from a natural oil using any one or combination of methods known in the art. Such methods include but are not limited to saponification, fat splitting, transesterification, esterification, hydrogenation (partial, selective, or full), isomerization, oxidation, and reduction. Representative non-limiting examples of natural oil derivatives include gums, phospholipids, soapstock, acidulated soapstock, distillate or distillate sludge, fatty acids and fatty acid alkyl ester (e.g. non-limiting examples such as 2-ethylhexyl ester), hydroxy substituted variations thereof of the natural oil. For example, the natural oil derivative may be a fatty acid methyl ester (“FAME”) derived from the glyceride of the natural oil. In some embodiments, a feedstock includes canola or soybean oil, as a non-limiting example, refined, bleached, and deodorized soybean oil (i.e., RBD soybean oil). Soybean oil typically comprises about 95% weight or greater (e.g., 99% weight or greater) triglycerides of fatty acids. Major fatty acids in the polyol esters of soybean oil include saturated fatty acids, as a non-limiting example, palm itic acid (hexadecanoic acid) and stearic acid (octadecanoic acid), and unsaturated fatty acids, as a non-limiting example, oleic acid (9-octadecenoic acid), linoleic acid (9, 12-octadecadienoic acid), and linolenic acid (9,12,15-octadecatrienoic acid).

As used herein, “metathesis catalyst” includes any catalyst or catalyst system that catalyzes an olefin metathesis reaction.

As used herein, “metathesize” or “metathesizing” refer to the reacting of a feedstock in the presence of a metathesis catalyst to form a “metathesized product” comprising new olefinic compounds, i.e., “metathesized” compounds. Metathesizing is not limited to any particular type of olefin metathesis, and may refer to cross-metathesis (i.e., co-metathesis), self-metathesis, ring-opening metathesis, ring-opening metathesis polymerizations (“ROMP”), ring-closing metathesis (“RCM”), and acyclic diene metathesis (“ADMET”). In some embodiments, metathesizing refers to reacting two triglycerides present in a natural feedstock (self-metathesis) in the presence of a metathesis catalyst, wherein each triglyceride has an unsaturated carbon-carbon double bond, thereby forming a new mixture of olefins and esters which may include a triglyceride dimer. Such triglyceride dimers may have more than one olefinic bond, thus higher oligomers also may form. Additionally, in some other embodiments, metathesizing may refer to reacting an olefin, such as ethylene, and a triglyceride in a natural feedstock having at least one unsaturated carbon-carbon double bond, thereby forming new olefinic molecules as well as new ester molecules (cross-metathesis).

As used herein, “olefin” or “olefins” refer to compounds having at least one unsaturated carbon-carbon double bond. In certain embodiments, the term “olefins” refers to a group of unsaturated carbon-carbon double bond compounds with different carbon lengths. Unless noted otherwise, the terms “olefin” or “olefins” encompasses “polyunsaturated olefins” or “poly-olefins,” which have more than one carbon-carbon double bond. As used herein, the term “monounsaturated olefins” or “mono-olefins” refers to compounds having only one carbon-carbon double bond. A compound having a terminal carbon-carbon double bond can be referred to as a “terminal olefin” or an “alpha-olefin,” while an olefin having a non-terminal carbon-carbon double bond can be referred to as an “internal olefin.” In some embodiments, the alpha-olefin is a terminal alkene, which is an alkene (as defined below) having a terminal carbon-carbon double bond. Additional carbon-carbon double bonds can be present.

The number of carbon atoms in any group or compound can be represented by the terms: “C_{z}”, which refers to a group of compound having z carbon atoms; and “C_{x-y}”, which refers to a group or compound containing from x to y, inclusive, carbon atoms. For example, “C₁₋₆ alkyl” represents an alkyl chain having from 1 to 6 carbon atoms and, for example, includes, but is not limited to, methyl, ethyl, n-propyl, isopropyl, isobutyl, n-butyl, sec-butyl, tert-butyl, isopentyl, n-pentyl, neopentyl, and n-hexyl. As a further example, a “C₄₋₁₀ alkene” refers to an alkene molecule having from 4 to 10 carbon atoms, and, for example, includes, but is not limited to, 1-butene, 2-butene, isobutene, 1-pentene, 1-hexene, 3-hexene, 1-heptene, 3-heptene, 1-octene, 4-octene, 1-nonene, 4-nonene, and 1-decene.

As used herein, the term “low-molecular-weight olefin” may refer to any one or combination of unsaturated straight, branched, or cyclic hydrocarbons in the C₂₋₁₄ range. Low-molecular-weight olefins include alpha-olefins, wherein the unsaturated carbon-carbon bond is present at one end of the compound. Low-molecular-weight olefins may also include dienes or trienes. Low-molecular-weight olefins may also include internal olefins or “low-molecular-weight internal olefins.” In certain embodiments, the low-molecular-weight internal olefin is in the C₄₋₁₄ range. Examples of low-molecular-weight olefins in the C₂₋₆ range include, but are not limited to: ethylene, propylene, 1-butene, 2-butene, isobutene, 1-pentene, 2-pentene, 3-pentene, 2-methyl-1-butene, 2-methyl-2-butene, 3-methyl-1-butene, cyclopentene, 1,4-pentadiene, 1-hexene, 2-hexene, 3-hexene, 4-hexene, 2-methyl-1-pentene, 3-methyl-1-pentene, 4-methyl-1-pentene, 2-methyl-2-pentene, 3-methyl-2-pentene, 4-methyl-2-pentene, 2-methyl-3-pentene, and cyclohexene. Non-limiting examples of low-molecular-weight olefins in the C₇₋₉ range include 1,4-heptadiene, 1-heptene, 3,6-nonadiene, 3-nonene, 1,4,7-octatriene. Other possible low-molecular-weight olefins include styrene and vinyl cyclohexane. In certain embodiments, it is preferable to use a mixture of olefins, the mixture comprising linear and branched low-molecular-weight olefins in the C₄₋₁₀ range. Olefins in the C₄₋₁₀ range can also be referred to as “short-chain olefins,” which can be either branched or unbranched. In one embodiments, it may be preferable to use a mixture of linear and branched C₄ olefins (i.e., combinations of: 1-butene, 2-butene, and/or isobutene). In other embodiments, a higher range of C₁₁₋₁₄ may be used.

In some instances, the olefin can be an “alkene,” which refers to a straight- or branched-chain non-aromatic hydrocarbon having 2 to 30 carbon atoms and one or more carbon-carbon double bonds, which may be optionally substituted, as herein further described, with multiple degrees of substitution being allowed. A “monounsaturated alkene” refers to an alkene having one carbon-carbon double bond, while a “polyunsaturated alkene” refers to an alkene having two or more carbon-carbon double bonds. A “lower alkene,” as used herein, refers to an alkene having from 2 to 10 carbon atoms.

As used herein, “ester” or “esters” refer to compounds having the general formula: R—COO—R′, wherein R and R′ denote any organic group (such as alkyl, aryl, or silyl groups) including those bearing heteroatom-containing substituent groups. In certain embodiments, R and R′ denote alkyl, alkenyl, aryl, or alcohol groups. In certain embodiments, the term “esters” may refer to a group of compounds with the general formula described above, wherein the compounds have different carbon lengths. In certain embodiments, the esters may be esters of glycerol, which is a trihydric alcohol. The term “glyceride” can refer to esters where one, two, or three of the —OH groups of the glycerol have been esterified.

It is noted that an olefin may also comprise an ester, and an ester may also comprise an olefin, if the R or R′ group in the general formula R—COO—R′ contains an unsaturated carbon-carbon double bond. Such compounds can be referred to as “unsaturated esters” or “olefin ester” or “olefinic ester compounds.” Further, a “terminal olefinic ester compound” may refer to an ester compound where R has an olefin positioned at the end of the chain. An “internal olefin ester” may refer to an ester compound where R has an olefin positioned at an internal location on the chain. Additionally, the term “terminal olefin” may refer to an ester or an acid thereof where R′ denotes hydrogen or any organic compound (such as an alkyl, aryl, or silyl group) and R has an olefin positioned at the end of the chain, and the term “internal olefin” may refer to an ester or an acid thereof where R′ denotes hydrogen or any organic compound (such as an alkyl, aryl, or silyl group) and R has an olefin positioned at an internal location on the chain.

As used herein, “acid,” “acids,” “carboxylic acid,” or “carboxylic acids” refer to compounds having the general formula: R—COOH, wherein R denotes any organic moiety (such as alkyl, aryl, or silyl groups), including those bearing heteroatom-containing substituent groups. In certain embodiments, R denotes alkyl, alkenyl, aryl, or alcohol groups. In certain embodiments, the term “acids” or “carboxylic acids” may refer to a group of compounds with the general formula described above, wherein the compounds have different carbon lengths.

As used herein, “alcohol” or “alcohols” refer to compounds having the general formula: R—OH, wherein R denotes any organic moiety (such as alkyl, aryl, or silyl groups), including those bearing heteroatom-containing substituent groups. In certain embodiments, R denotes alkyl, alkenyl, aryl, or alcohol groups. In certain embodiments, the term “alcohol” or “alcohols” may refer to a group of compounds with the general formula described above, wherein the compounds have different carbon lengths. As used herein, the term “alkanol” refers to alcohols where R is an alkyl group.

As used herein, “hydrocarbon” refers to an organic group composed of carbon and hydrogen, which can be saturated or unsaturated, and can include aromatic groups. The term “hydrocarbyl” refers to a monovalent or polyvalent (e.g., divalent or higher) hydrocarbon moiety. In some instances, a divalent hydrocarbyl group can be referred to as a “hydrocarbylene” group.

As used herein, “alkyl” refers to a straight or branched chain saturated hydrocarbon having 1 to 30 carbon atoms, which may be optionally substituted, as herein further described, with multiple degrees of substitution being allowed. Examples of “alkyl,” as used herein, include, but are not limited to, methyl, ethyl, n-propyl, isopropyl, isobutyl, n-butyl, sec-butyl, tert-butyl, isopentyl, n-pentyl, neopentyl, n-hexyl, and 2-ethylhexyl. The number of carbon atoms in an alkyl group is represented by the phrase “C_{x-y} alkyl,” which refers to an alkyl group, as herein defined, containing from x to y, inclusive, carbon atoms. Thus, “C₁₋₆ alkyl” represents an alkyl chain having from 1 to 6 carbon atoms and, for example, includes, but is not limited to, methyl, ethyl, n-propyl, isopropyl, isobutyl, n-butyl, sec-butyl, tert-butyl, isopentyl, n-pentyl, neopentyl, and n-hexyl. In some instances, the “alkyl” group can be divalent, in which case the group can alternatively be referred to as an “alkylene” group. Also, in some instances, one or more of the carbon atoms in the alkyl or alkylene group can be replaced by a heteroatom (e.g., selected from nitrogen, oxygen, or sulfur, including N-oxides, sulfur oxides, and sulfur dioxides, where feasible), and is referred to as a “heteroalkyl” or “heteroalkylene” group, respectively. Non-limiting examples include “oxyalkyl” or “oxyalkylene” groups, which include groups of the following formulas: -[-(alkylene)-O-]ₓ-alkyl, or —O-[-(alkylene)-O-]ₓ-alkyl, -[-(alkylene)-O-]ₓ-alkylene-, respectively, where x is 1 or more, such as 1, 2, 3, 4, 5, 6, 7, or 8.

As used herein, “alkenyl” refers to a straight or branched chain non-aromatic hydrocarbon having 2 to 30 carbon atoms and having one or more carbon-carbon double bonds, which may be optionally substituted, as herein further described, with multiple degrees of substitution being allowed. Examples of “alkenyl,” as used herein, include, but are not limited to, ethenyl, 2-propenyl, 2-butenyl, and 3-butenyl. The number of carbon atoms in an alkenyl group is represented by the phrase “C_{x-y} alkenyl,” which refers to an alkenyl group, as herein defined, containing from x to y, inclusive, carbon atoms. Thus, “C₂₋₆ alkenyl” represents an alkenyl chain having from 2 to 6 carbon atoms and, for example, includes, but is not limited to, ethenyl, 2-propenyl, 2-butenyl, and 3-butenyl. In some instances, the “alkenyl” group can be divalent, in which case the group can alternatively be referred to as an “alkenylene” group. Also, in some instances, one or more of the carbon atoms in the alkyl or alkenylene group can be replaced by a heteroatom (e.g., selected from nitrogen, oxygen, or sulfur, including N-oxides, sulfur oxides, and sulfur dioxides, where feasible), and is referred to as a “heteroalkenyl” or “heteroalkenylene” group, respectively.

As used herein, “cycloalkyl” refers to an aliphatic saturated or unsaturated hydrocarbon ring system having 1 to 20 carbon atoms, which may be optionally substituted, as herein further described, with multiple degrees of substitution being allowed. In some embodiments, the term refers only to saturated hydrocarbon ring systems, substituted as indicated above. Examples of “cycloalkyl,” as used herein, include, but are not limited to, cyclopropyl, cyclobutyl, cyclopentyl, cyclohexyl, cyclohexenyl, cycloheptyl, cyclooctyl, adamantyl, and the like. The number of carbon atoms in a cycloalkyl group is represented by the phrase “C_{x-y} cycloalkyl,” which refers to a cycloalkyl group, as herein defined, containing from x to y, inclusive, carbon atoms. Thus, “C₃₋₁₀ cycloalkyl” represents a cycloalkyl having from 3 to 10 carbon atoms and, for example, includes, but is not limited to, cyclopropyl, cyclobutyl, cyclopentyl, cyclohexyl, cyclohexenyl, cycloheptyl, cyclooctyl, and adamantyl. In some instances, the “cycloalkyl” group can be divalent, in which case the group can alternatively be referred to as a “cycloalkylene” group. Cycloalkyl and cycloalkylene groups can also be referred to herein as “carbocyclic rings.” Also, in some instances, one or more of the carbon atoms in the cycloalkyl or cycloalkylene group can be replaced by a heteroatom (e.g., selected independently from nitrogen, oxygen, silicon, or sulfur, including N-oxides, sulfur oxides, and sulfur dioxides, where feasible), and is referred to as a “heterocyclyl” or “heterocyclylene” group, respectively. The term “heterocyclic ring” can also be used interchangeable with either of these terms. In some embodiments, the cycloalkyl and heterocyclyl groups are fully saturated. In some other embodiments, the cycloalkyl and heterocyclyl groups can contain one or more carbon-carbon double bonds.

As used herein, “halogen” or “halo” refers to a fluorine, chlorine, bromine, and/or iodine atom. In some embodiments, the terms refer to fluorine or chlorine.

As used herein, a “UV-stabilizing residue” is a moiety that, when used in combination with a UV-absorbing compound, improves the useful lifetime of the UV-absorbing compound (i.e., a chemical filter of UV radiation) under conditions of UV light exposure.

As used herein, “substituted” refers to substitution of one or more hydrogen atoms of the designated moiety with the named substituent or substituents, multiple degrees of substitution being allowed unless otherwise stated, provided that the substitution results in a stable or chemically feasible compound. A stable compound or chemically feasible compound is one in which the chemical structure is not substantially altered when kept at a temperature from about −80° C. to about +40° C., in the absence of moisture or other chemically reactive conditions, for at least a week, or a compound which maintains its integrity long enough to be useful for therapeutic or prophylactic administration to a patient. As used herein, the phrases “substituted with one or more . . . ” or “substituted one or more times . . . ” refer to a number of substituents that equals from one to the maximum number of substituents possible based on the number of available bonding sites, provided that the above conditions of stability and chemical feasibility are met.

As used herein, the term “fatty acid residue” refers to —C(O)—R, where R is an organic group, such as an alkyl group. For example, —C(O)—(CH₂)₁₆—CH₃ is a stearic acid residue.

As used herein, “mix” or “mixed” or “mixture” refers broadly to any combining of two or more compositions. The two or more compositions need not have the same physical state; thus, solids can be “mixed” with liquids, e.g., to form a slurry, suspension, or solution. Further, these terms do not require any degree of homogeneity or uniformity of composition. This, such “mixtures” can be homogeneous or heterogeneous, or can be uniform or non-uniform. Further, the terms do not require the use of any particular equipment to carry out the mixing, such as an industrial mixer.

As used herein, “optionally” means that the subsequently described event(s) may or may not occur. In some embodiments, the optional event does not occur. In some other embodiments, the optional event does occur one or more times.

As used herein, “comprise” or “comprises” or “comprising” or “comprised of” refer to groups that are open, meaning that the group can include additional members in addition to those expressly recited. For example, the phrase, “comprises A” means that A must be present, but that other members can be present too. The terms “include,” “have,” and “composed of” and their grammatical variants have the same meaning. In contrast, “consist of” or “consists of” or “consisting of” refer to groups that are closed. For example, the phrase “consists of A” means that A and only A is present.

As used herein, “or” is to be given its broadest reasonable interpretation, and is not to be limited to an either/or construction. Thus, the phrase “comprising A or B” means that A can be present and not B, or that B is present and not A, or that A and B are both present. Further, if A, for example, defines a class that can have multiple members, e.g., A₁ and A₂, then one or more members of the class can be present concurrently.

In some instances herein, organic compounds are described using the “line structure” methodology, where chemical bonds are indicated by a line, where the carbon atoms are not expressly labeled, and where the hydrogen atoms covalently bound to carbon (or the C—H bonds) are not shown at all. For example, by that convention, the formula [symbol] represents n-propane. In some instances herein, a squiggly bond is used to show the compound can have any one of two or more isomers. For example, the structure

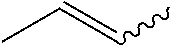

can reter to (E)-2-butene or (Z)-2-butene.

As used herein, the various functional groups represented will be understood to have a point of attachment at the functional group having the hyphen or dash (—) or an asterisk (*). In other words, in the case of —CH₂CH₂CH₃, it will be understood that the point of attachment is the CH₂ group at the far left. If a group is recited without an asterisk or a dash, then the attachment point is indicated by the plain and ordinary meaning of the recited group.

As used herein, multi-atom bivalent species are to be read from left to right. For example, if the specification or claims recite A-D-E and D is defined as —OC(O)—, the resulting group with D replaced is: A-OC(O)-E and not A-C(O)O-E.

Other terms are defined in other portions of this description, even though not included in this subsection.

### UV-Stabilizing Compounds

In certain aspects, the disclosure provides compounds of formula (I):

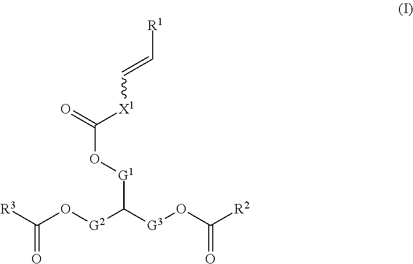

wherein: X¹ is C₁₋₂₀ alkylene, C₂₋₂₀ alkenylene, C₁₋₂₀ heteroalkylene, or C₂₋₂₀ heteroalkenylene, each of which is optionally substituted one or more times by R^{x}; two of G¹, G², and G³ are —CH₂—, and one of G¹, G², and G³ is a direct bond; R¹ is a moiety comprising a UV-stabilizing residue, such as —X⁴—R⁸, wherein X⁴ is C₁₋₂₀ hydrocarbylene, which is optionally substituted one or more times by R^{x}, and R⁸ is a UV-stabilizing residue; R² is C₁₋₂₄ alkyl, C₂₋₂₄ alkenyl, C₁₋₂₄ heteroalkyl, or C₂₋₂₄ heteroalkenyl, each of which is optionally substituted one or more times by R^{x}, or R² is a moiety of the following formula:

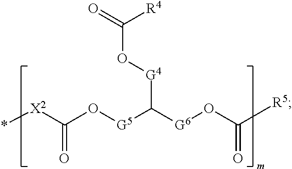

R³ is C₁₋₂₄ alkyl, C₂₋₂₄ alkenyl, C₁₋₂₄ heteroalkyl, or C₂₋₂₄ heteroalkenyl, each of which is optionally substituted one or more times by R^{x}, or R³ is a moiety of the following formula:

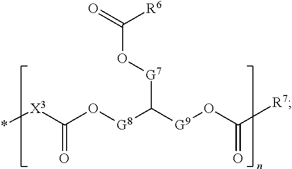

X² and X³ are independently C₁₋₃₀ alkylene, C₂₋₃₀ alkenylene, C₁₋₃₀ heteroalkylene, or C₂₋₃₀ heteroalkenylene, each of which is optionally substituted one or more times by R^{x}; two of G⁴, G⁵, and G⁶ are —CH₂—, and one of G⁴, G⁵, and G⁶ is a direct bond, wherein, if m is greater than 1, these assignments are made independently for each repeating unit; two of G⁷, G⁸, and G⁹ are —CH₂—, and one of G⁷, G⁸, and G⁹ is a direct bond, wherein, if n is greater than 1, these assignments are made independently for each repeating unit; R⁴ and R⁶ are independently C₁₋₂₄ alkyl, C₂₋₂₄ alkenyl, C₁₋₂₄ heteroalkyl, or C₂₋₂₄ heteroalkenyl, each of which is optionally substituted one or more times by R^{x}, or are independently a moiety comprising a UV-stabilizing residue, such as —X¹⁴—R¹⁸, wherein X¹⁴ is C₁₋₂₀ hydrocarbylene, which is optionally substituted one or more times by R^{x}, and R¹⁸ is a UV-stabilizing residue; R⁵ and R⁷ are independently C₁₋₂₄ alkyl, C₂₋₂₄ alkenyl, C₁₋₂₄ heteroalkyl, or C₂₋₂₄ heteroalkenyl, each of which is optionally substituted one or more times by R^{x}; m and n are independently 0, 1, 2, 3, 4, or 5; and R^{x} is a halogen atom, —OH, —NH₂, C₁₋₆ alkyl, C₁₋₆ heteroalkyl, C₂₋₆ alkenyl, or C₂₋₆ heteroalkenyl.

In some embodiments of any of the aforementioned embodiments, G¹ and G² are —CH₂— and G³ is a direct bond. In some embodiments of any of the aforementioned embodiments, G¹ and G³ are —CH₂— and G² is a direct bond. In some embodiments of any of the aforementioned embodiments, G² and G³ are —CH₂— and G¹ is a direct bond.

In some embodiments of any of the aforementioned embodiments, X¹ is C₁₋₂₀ alkylene or C₂₋₂₀ alkenylene, each of which is optionally substituted one or more times by substituents selected from the group consisting of —OH and C₁₋₆ oxyalkyl. In some such embodiments, X¹ is C₁₋₂₀ alkylene or C₂₋₂₀ alkenylene. In some further embodiments, X¹ is —(CH₂)₅—, —(CH₂)₆—, —(CH₂)₇—, —(CH₂)₈—, or —(CH₂)₉—. In some further such embodiments, X¹ is —(CH₂)₇—. In some embodiments, X¹ is —(CH₂)₇—CH═CH—CH₂— or —(CH₂)₇—CH═CH—CH₂—CH═CH—CH₂—. In some embodiments, X¹ is —(CH₂)₇—CH═CH—CH₂—. In some embodiments, X¹ is —(CH₂)₇—CH═CH—CH₂—CH═CH—CH₂—.

In some embodiments of any of the aforementioned embodiments, R¹ is —X⁴—R⁸. In some such embodiments, X⁴ is C₁₋₂₀ hydrocarbylene, which is optionally substituted one or more times by R^{x}. In some such embodiments, X⁴ is C₁₋₂₀ alkylene, C₂₋₂₀ alkenylene, C₁₋₂₀ heteroalkylene, or C₂₋₂₀ heteroalkenylene, each of which is optionally substituted one or more times by R^{x}. In some such embodiments, X⁴ is C₁₋₂₀ alkylene or C₂₋₂₀ alkenylene, each of which is optionally substituted one or more times by substituents selected from the group consisting of —OH and C₁₋₆ oxyalkyl. In some further such embodiments, X⁴ is C₁₋₂₀ alkylene or C₂₋₂₀ alkenylene. In some further such embodiments, X⁴ is C₁₋₆ alkylene. In some further embodiments, X⁴ is —(CH₂)—, —(CH₂)₂—, —(CH₂)₃—, —(CH₂)₄—, —(CH₂)₅—, —(CH₂)₆—, —(CH₂)₇—, —(CH₂)₈—, —(CH₂)₉—, —(CH₂)₁₀—, —(CH₂)₁₁—, —(CH₂)₁₂—, —(CH₂)₁₃—, or —(CH₂)₁₄—. In some further such embodiments, X⁴ is —(CH₂)—. In some such embodiments, X⁴ is -(phenylene)-CH═. In some such embodiments, X⁴ is ═CH-(phenylene)-.

In some embodiments of any of the aforementioned embodiments, the UV-stabilizing residue, for example, R⁸, can be any suitable moiety that can improve the useful lifetime of a UV chemical filter. In some such embodiments, the UV-stabilizing residue can also be a moiety that absorbs UV radiation, such as UV-A and/or UV-B radiation. For example, in some embodiments, the UV-stabilizing residue is a derivative the UV-chemical filter. With reference to this context, the term “derivative” refers to moieties that have undergone simple transformations to facilitate their linkage as part of the compound of formula (I), e.g., via X⁴ (in any of the aforementioned embodiments). Thus, in some embodiments, the UV-stabilizing residue (e.g., R⁸) is a derivative of a UV chemical filter, such as a chemical filter that absorbs UV-A and/or UV-B radiation.

The UV-stabilizing residue can have any acceptable molecular weight. Thus, in some embodiments, the UV-stabilizing residue is a polymer. In some other embodiments, the UV-stabilizing residue is a small molecule, such as a moiety having a molecular weight of no more than 2000 Da, or no more than 1500 Da, or no more than 1000 Da, or no more than 750 Da, or no more than 500 Da.

In some embodiments, the UV-stabilizing residue operates by accepting a photon from the UV-chemical filter. Thus, the UV-stabilizing residue absorbs UV radiation at approximately the same wavelength as the UV chemical filter that is being stabilized. Therefore, some UV-stabilizing residues can stabilize UV-A radiation, UV-B radiation, or both UV-A and UV-B radiation, e.g., by being able to absorb a photon having a wavelength in that range(s) of UV-A and/or UV-B radiation. In some embodiments, the UV-stabilizing residue is a moiety that stabilizes chemical filters of UV-A radiation, e.g., by absorbing a photon of UV-A radiation from the chemical filter. In some embodiments, the UV-stabilizing residue is a moiety that stabilizes chemical filters of UV-B radiation, e.g., by absorbing a photon of UV-B radiation from the chemical filter.

Any suitable type of stabilizing residue can be used, such as derivatives of known UV-stabilizing compounds. In some embodiments, the UV-stabilizing residue, e.g., R⁸, is a dinaphthalate, a salicylate, a crylene, a fluorine, a camphor, a syringlidene malonate, a polysilicone, a polyester, a benzotriazole, a triazine, a methoxycinnamate, a sulfonic acid, a benzone, or a benzoic acid.

In some embodiments, R¹ is:

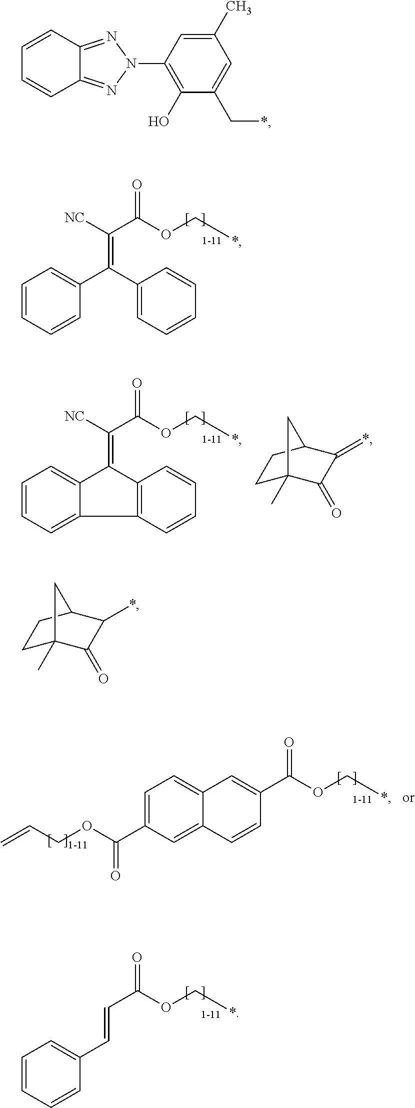

In some embodiments of any of the aforementioned embodiments, R² is C₁₋₂₄ alkyl, C₂₋₂₄ alkenyl, C₁₋₂₄ heteroalkyl, or C₂₋₂₄ heteroalkenyl, each of which is optionally substituted one or more times by R^{x}. In some such embodiments, R² is methyl, ethyl, propyl, isopropyl, butyl, isobutyl, sec-butyl, or tert-butyl. In some such embodiments, R² is —(CH₂)₁₂—CH₃, —(CH₂)₁₄—CH₃, —(CH₂)₁₆—CH₃, —(CH₂)₇—CH═CH—(CH₂)₇—CH₃, —(CH₂)₇—CH═CH—CH₂—CH═CH—(CH₂)₄—CH₃, or —(CH₂)₇—CH═CH—CH₂—CH═CH—CH₂—CH═CH—CH₂—CH₃.

In some embodiments of any of the aforementioned embodiments, R² is a moiety of the following formula:

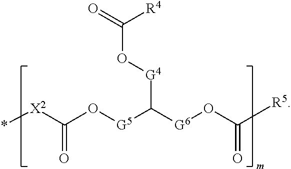

In some embodiments of any of the aforementioned embodiments, m is 1, or m is 2, or m is 3, or m is 4, or m is 5.

In some embodiments of any of the aforementioned embodiments, X² is independently C₁₆₋₂₈ alkylene or C₁₆₋₂₈ alkenylene, each of which is optionally substituted one or more times by R^{x}. Note that, in this context, “independently” means that, if m is 2 or more, the identity of each X² is selected independently of the identity of another X². In some such embodiments, X² is independently —(CH₂)₁₆—, —(CH₂)₁₉—, —(CH₂)₂₂—, —(CH₂)₂₅—, or —(CH₂)₂₈—. In some such embodiments, X² is —(CH₂)₁₆—. In some such embodiments, X² is independently —(CH₂)₇—CH═CH—(CH₂)₇—, —(CH₂)₇—CH═CH—CH₂—CH═CH—(CH₂)₇—, —(CH₂)₇—CH═CH—CH₂—CH═CH—CH₂—CH═CH—(CH₂)₇—, —(CH₂)₇—CH═CH—CH₂—CH═CH—CH₂—CH═CH—CH₂—CH═CH—(CH₂)₇—, or —(CH₂)₇—CH═CH—CH₂—CH═CH—CH₂—CH═CH—CH₂—CH═CH—CH₂—CH═CH—(CH₂)₇—. In some such embodiments, at least one X² is —(CH₂)₇—CH═CH—(CH₂)₇—, —(CH₂)₇—CH═CH—CH₂—CH═CH—(CH₂)₇—, —(CH₂)₇—CH═CH—CH₂—CH═CH—CH₂—CH═CH—(CH₂)₇—, —(CH₂)₇—CH═CH—CH₂—CH═CH—CH₂—CH═CH—CH₂—CH═CH—(CH₂)₇—, or —(CH₂)₇—CH═CH—CH₂—CH═CH—CH₂—CH═CH—CH₂—CH═CH—CH₂—CH═CH—(CH₂)₇—. In some such embodiments, at least one X² is —(CH₂)₇—CH═CH—(CH₂)₇—.

In some embodiments of any of the aforementioned embodiments, at least one R⁴ is C₁₋₂₄ alkyl or C₂₋₂₄ alkenyl, each of which is optionally substituted one or more times by R^{x}. In some such embodiments, at least one R⁴ is methyl, ethyl, propyl, isopropyl, butyl, isobutyl, sec-butyl, or tert-butyl. In some such embodiments, at least one R⁴ is —(CH₂)₁₂—CH₃, —(CH₂)₁₄—CH₃, —(CH₂)₁₆—CH₃, —(CH₂)₇—CH═CH—(CH₂)₇—CH₃, —(CH₂)₇—CH═CH—CH₂—CH═CH—(CH₂)₄—CH₃, or —(CH₂)₇—CH═CH—CH₂—CH═CH—CH₂—CH═CH—CH₂—CH₃.

In some embodiments of any of the aforementioned embodiments, at least one R⁴ is a moiety comprising a UV-stabilizing residue (according to any of the embodiments described above for R¹).

In some such embodiments, R⁴ is —X¹⁴—R¹⁸. In some such embodiments, X¹⁴ is C₁₋₂₀ hydrocarbylene, which is optionally substituted one or more times by R^{x}. In some such embodiments, X¹⁴ is C₁₋₂₀ alkylene, C₂₋₂₀ alkenylene, C₁₋₂₀ heteroalkylene, or C₂₋₂₀ heteroalkenylene, each of which is optionally substituted one or more times by R^{x}. In some such embodiments, X¹⁴ is C₁₋₂₀ alkylene or C₂₋₂₀ alkenylene, each of which is optionally substituted one or more times by substituents selected from the group consisting of —OH and C₁₋₆ oxyalkyl. In some further such embodiments, X¹⁴ is C₁₋₂₀ alkylene or C₂₋₂₀ alkenylene. In some further such embodiments, X¹⁴ is C₁₋₆ alkylene. In some further embodiments, X¹⁴ is —(CH₂)—, —(CH₂)₂—, —(CH₂)₃—, —(CH₂)₄—, or —(CH₂)₅—, —(CH₂)₆—, —(CH₂)₇—, —(CH₂)₈—, or —(CH₂)₉—, —(CH₂)₁₀—, —(CH₂)₁₁—, —(CH₂)₁₂—, or —(CH₂)₁₃—, or —(CH₂)₁₄—. In some further such embodiments, X¹⁴ is —(CH₂)₇—.

In some embodiments of any of the aforementioned embodiments, the UV-stabilizing residue, for example, R¹⁸, can be any suitable moiety that can improve the useful lifetime of a UV chemical filter. In some such embodiments, the UV-stabilizing residue can also be a moiety that absorbs UV radiation, such as UV-A and/or UV-B radiation. For example, in some embodiments, the UV-stabilizing residue is a derivative the UV-chemical filter. With reference to this context, the term “derivative” refers to moieties that have undergone simple transformations to facilitate their linkage as part of the compound of formula (I), e.g., via X¹⁴ (in any of the aforementioned embodiments). Thus, in some embodiments, the UV-stabilizing residue (e.g., R¹⁸) is a derivative of a UV chemical filter, such as a chemical filter that absorbs UV-A and/or UV-B radiation.

The UV-stabilizing residue can have any acceptable molecular weight. Thus, in some embodiments, the UV-stabilizing residue is a polymer. In some other embodiments, the UV-stabilizing residue is a small molecule, such as a moiety having a molecular weight of no more than 2000 Da, or no more than 1500 Da, or no more than 1000 Da, or no more than 750 Da, or no more than 500 Da.

In some embodiments, the UV-stabilizing residue operates by accepting a photon from the UV-chemical filter. Thus, the UV-stabilizing residue absorbs UV radiation at approximately the same wavelength as the UV chemical filter that is being stabilized. Therefore, some UV-stabilizing residues can stabilize UV-A radiation, UV-B radiation, or both UV-A and UV-B radiation, e.g., by being able to absorb a photon having a wavelength in that range(s) of UV-A and/or UV-B radiation. In some embodiments, the UV-stabilizing residue is a moiety that stabilizes chemical filters of UV-A radiation, e.g., by absorbing a photon of UV-A radiation from the chemical filter. In some embodiments, the UV-stabilizing residue is a moiety that stabilizes chemical filters of UV-B radiation, e.g., by absorbing a photon of UV-B radiation from the chemical filter.

Any suitable type of stabilizing residue can be used, such as derivatives of known UV-stabilizing compounds. In some embodiments, the UV-stabilizing residue, e.g., R⁸, is a dinaphthalate, a salicylate, a crylene, a fluorine, a camphor, a syringlidene malonate, a polysilicone, a polyester, a benzotriazole, a triazine, a methoxycinnamate, a sulfonic acid, a benzone, or a benzoic acid.

In some embodiments, R¹⁸ is:

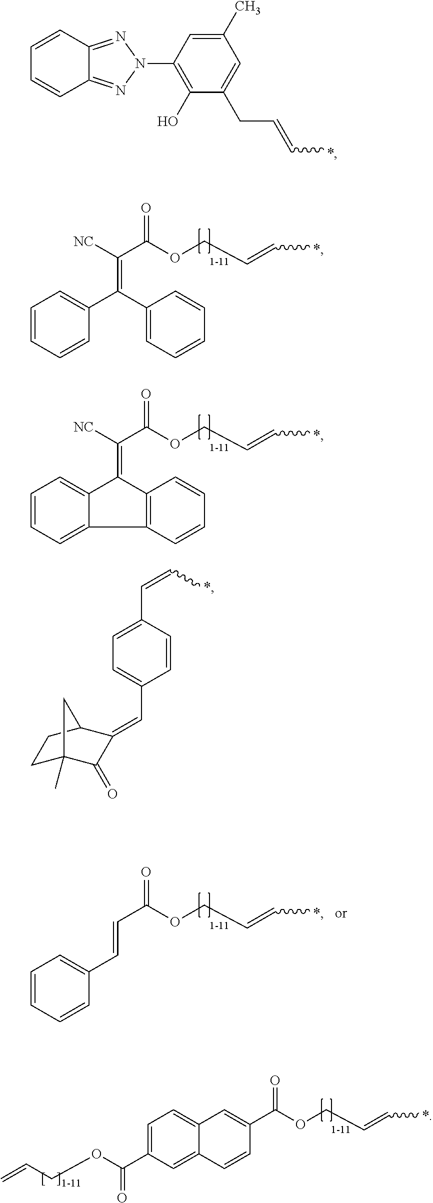

In some embodiments of any of the aforementioned embodiments, in at least one instance, G⁴ and G⁵ are —CH₂— and G⁶ is a direct bond. In some embodiments of any of the aforementioned embodiments, in at least one instance, G⁴ and G⁶ are —CH₂— and G⁵ is a direct bond. In some embodiments of any of the aforementioned embodiments, in at least one instance, G⁵ and G⁶ are —CH₂— and G⁴ is a direct bond.

In some embodiments of any of the aforementioned embodiments, R⁵ is C₁₋₂₄ alkyl or C₂₋₂₄ alkenyl, each of which is optionally substituted one or more times by R^{x}. In some such embodiments, R⁵ is methyl, ethyl, propyl, isopropyl, butyl, isobutyl, sec-butyl, or tert-butyl. In some such embodiments, R⁵ is —(CH₂)₁₂—CH₃, —(CH₂)₁₄—CH₃, —(CH₂)₁₆—CH₃, —(CH₂)₇—CH═CH—(CH₂)₇—CH₃, —(CH₂)₇—CH═CH—CH₂—CH═CH—(CH₂)₄—CH₃, or —(CH₂)₇—CH═CH—CH₂—CH═CH—CH₂—CH═CH—CH₂—CH₃.

In some embodiments of any of the aforementioned embodiments, R³ is C₁₋₂₄ alkyl, C₂₋₂₄ alkenyl, C₁₋₂₄ heteroalkyl, or C₂₋₂₄ heteroalkenyl, each of which is optionally substituted one or more times by R^{x}. In some such embodiments, R³ is methyl, ethyl, propyl, isopropyl, butyl, isobutyl, sec-butyl, or tert-butyl. In some such embodiments, R³ is —(CH₂)₁₂—CH₃, —(CH₂)₁₄—CH₃, —(CH₂)₁₆—CH₃, —(CH₂)₇—CH═CH—(CH₂)₇—CH₃, —(CH₂)₇—CH═CH—CH₂—CH═CH—(CH₂)₄—CH₃, or —(CH₂)₇—CH═CH—CH₂—CH═CH—CH₂—CH═CH—CH₂—CH₃.

In some embodiments of any of the aforementioned embodiments, R³ is a moiety of the following formula:

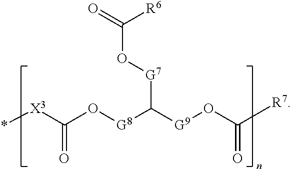

In some embodiments of any of the aforementioned embodiments, n is 1, or n is 2, or n is 3, or n is 4, or n is 5.

In some embodiments of any of the aforementioned embodiments, X³ is independently C₁₆₋₂₈ alkylene or C₁₆₋₂₈ alkenylene, each of which is optionally substituted one or more times by R^{x}. Note that, in this context, “independently” means that, if n is 2 or more, the identity of each X³ is selected independently of the identity of another X³. In some such embodiments, X³ is independently —(CH₂)₁₆—, —(CH₂)₁₉—, —(CH₂)₂₂—, —(CH₂)₂₅—, or —(CH₂)₂₈—. In some such embodiments, X³ is —(CH₂)₁₆—. In some such embodiments, X³ is independently —(CH₂)₇—CH═CH—(CH₂)₇—, —(CH₂)₇—CH═CH—CH₂—CH═CH—(CH₂)₇—, —(CH₂)₇—CH═CH—CH₂—CH═CH—CH₂—CH═CH—(CH₂)₇—, —(CH₂)₇—CH═CH—CH₂—CH═CH—CH₂—CH═CH—CH₂—CH═CH—(CH₂)₇—, or —(CH₂)₇—CH═CH—CH₂—CH═CH—CH₂—CH═CH—CH₂—CH═CH—CH₂—CH═CH—(CH₂)₇—. In some such embodiments, at least one X³ is —(CH₂)₇—CH═CH—(CH₂)₇—, —(CH₂)₇—CH═CH—CH₂—CH═CH—(CH₂)₇—, —(CH₂)₇—CH═CH—CH₂—CH═CH—CH₂—CH═CH—(CH₂)₇—, —(CH₂)₇—CH═CH—CH₂—CH═CH—CH₂—CH═CH—CH₂—CH═CH—(CH₂)₇—, or —(CH₂)₇—CH═CH—CH₂—CH═CH—CH₂—CH═CH—CH₂—CH═CH—CH₂—CH═CH—(CH₂)₇—. In some such embodiments, at least one X³ is —(CH₂)₇—CH═CH—(CH₂)₇—.

In some embodiments of any of the aforementioned embodiments, at least one R⁶ is C₁₋₂₄ alkyl or C₂₋₂₄ alkenyl, each of which is optionally substituted one or more times by R^{x}. In some such embodiments, at least one R⁶ is methyl, ethyl, propyl, isopropyl, butyl, isobutyl, sec-butyl, or tert-butyl. In some such embodiments, at least one R⁶ is —(CH₂)₁₂—CH₃, —(CH₂)₁₄—CH₃, —(CH₂)₁₆—CH₃, —(CH₂)₇—CH═CH—(CH₂)₇—CH₃, —(CH₂)₇—CH═CH—CH₂—CH═CH—(CH₂)₄—CH₃, or —(CH₂)₇—CH═CH—CH₂—CH═CH—CH₂—CH═CH—CH₂—CH₃.

In some embodiments of any of the aforementioned embodiments, at least one R⁶ is a moiety comprising a UV-stabilizing residue (according to any of the embodiments described above for R¹ and/or R⁴).

In some such embodiments, R⁶ is —X¹⁴—R¹⁸. In some such embodiments, X¹⁴ is C₁₋₂₀ hydrocarbylene, which is optionally substituted one or more times by R^{x}. In some such embodiments, X¹⁴ is C₁₋₂₀ alkylene, C₂₋₂₀ alkenylene, C₁₋₂₀ heteroalkylene, or C₂₋₂₀ heteroalkenylene, each of which is optionally substituted one or more times by R^{x}. In some such embodiments, X¹⁴ is C₁₋₂₀ alkylene or C₂₋₂₀ alkenylene, each of which is optionally substituted one or more times by substituents selected from the group consisting of —OH and C₁₋₆ oxyalkyl. In some further such embodiments, X¹⁴ is C₁₋₂₀ alkylene or C2-20 alkenylene. In some further such embodiments, X¹⁴ is C₁₋₆ alkylene. In some further embodiments, X¹⁴ is —(CH₂)—, —(CH₂)₂—, —(CH₂)₃—, —(CH₂)₄—, —(CH₂)₅—, —(CH₂)₆—, —(CH₂)₇—, —(CH₂)₈—, —(CH₂)₉—, —(CH₂)₁₀—, —(CH₂)₁₁—, —(CH₂)₁₂—, —(CH₂)₁₃—, or —(CH₂)₁₄—. In some further such embodiments, X¹⁴ is —(CH₂)₇—.

In some embodiments of any of the aforementioned embodiments, the UV-stabilizing residue, for example, R¹⁸, can be any suitable moiety that can improve the useful lifetime of a UV chemical filter. In some such embodiments, the UV-stabilizing residue can also be a moiety that absorbs UV radiation, such as UV-A and/or UV-B radiation. For example, in some embodiments, the UV-stabilizing residue is a derivative the UV-chemical filter. With reference to this context, the term “derivative” refers to moieties that have undergone simple transformations to facilitate their linkage as part of the compound of formula (I), e.g., via X¹⁴ (in any of the aforementioned embodiments). Thus, in some embodiments, the UV-stabilizing residue (e.g., R¹⁸) is a derivative of a UV chemical filter, such as a chemical filter that absorbs UV-A and/or UV-B radiation.

The UV-stabilizing residue can have any acceptable molecular weight. Thus, in some embodiments, the UV-stabilizing residue is a polymer. In some other embodiments, the UV-stabilizing residue is a small molecule, such as a moiety having a molecular weight of no more than 2000 Da, or no more than 1500 Da, or no more than 1000 Da, or no more than 750 Da, or no more than 500 Da.

In some embodiments, the UV-stabilizing residue operates by accepting a photon from the UV-chemical filter. Thus, the UV-stabilizing residue absorbs UV radiation at approximately the same wavelength as the UV chemical filter that is being stabilized. Therefore, some UV-stabilizing residues can stabilize UV-A radiation, UV-B radiation, or both UV-A and UV-B radiation, e.g., by being able to absorb a photon having a wavelength in that range(s) of UV-A and/or UV-B radiation. In some embodiments, the UV-stabilizing residue is a moiety that stabilizes chemical filters of UV-A radiation, e.g., by absorbing a photon of UV-A radiation from the chemical filter. In some embodiments, the UV-stabilizing residue is a moiety that stabilizes chemical filters of UV-B radiation, e.g., by absorbing a photon of UV-B radiation from the chemical filter.

Any suitable type of stabilizing residue can be used, such as derivatives of known UV-stabilizing compounds. In some embodiments, the UV-stabilizing residue, e.g., R⁸, is a dinaphthalate, a salicylate, a crylene, a fluorine, a camphor, a syringlidene malonate, a polysilicone, a polyester, a benzotriazole, a triazine, a methoxycinnamate, a sulfonic acid, a benzone, or a benzoic acid.

In some embodiments, R¹⁸ is:

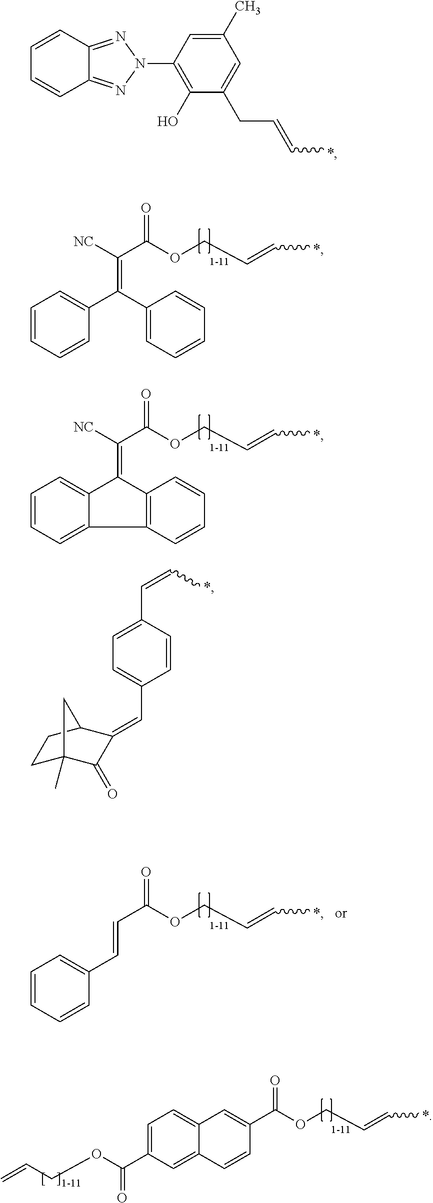

In some embodiments of any of the aforementioned embodiments, in at least one instance, G⁷ and G⁸ are —CH₂— and G⁹ is a direct bond. In some embodiments of any of the aforementioned embodiments, in at least one instance, G⁷ and G⁹ are —CH₂— and G⁸ is a direct bond. In some embodiments of any of the aforementioned embodiments, in at least one instance, G⁸ and G⁹ are —CH₂— and G⁷ is a direct bond.

In some embodiments of any of the aforementioned embodiments, R⁷ is C₁₋₂₄ alkyl or C₂₋₂₄ alkenyl, each of which is optionally substituted one or more times by R^{x}. In some such embodiments, R⁷ is methyl, ethyl, propyl, isopropyl, butyl, isobutyl, sec-butyl, or tert-butyl. In some such embodiments, R⁷ is —(CH₂)₁₂—CH₃, —(CH₂)₁₄—CH₃, —(CH₂)₁₆—CH₃, —(CH₂)₇—CH═CH—(CH₂)₇—CH₃, —(CH₂)₇—CH═CH—CH₂—CH═CH—(CH₂)₄—CH₃, or —(CH₂)₇—CH═CH—CH₂—CH═CH—CH₂—CH═CH—CH₂—CH₃.

In some embodiments of any of the aforementioned embodiments, the sum of m and n (i.e., m+n) is 0. In some other embodiments of any of the aforementioned embodiments, the sum of m and n is at least 1. In some such embodiments, the sum of m and n is 1, or is 2, or is 3, or is 4, or is 5, or is 6.

Further, in some embodiments, the compounds of formula (I) can be partially or fully hydrogenated following their formation using standard hydrogenation techniques known in the art.

In a certain aspects, the disclosure provides com pounds formed by reacting one or more unsaturated glycerides with a compound of formula (II)

HC═CH—R²¹ (II)

in the presence of a metathesis catalyst, wherein R²¹ is a moiety comprising a UV-stabilizing residue.

Any suitable unsaturated glycerides can be used. In some embodiments, the unsaturated glycerides comprise at least one unsaturated fatty acid residue, such as, for example, oleic acid residues, a linoleic acid residues, and/or a linolenic acid residue. Such glycerides can have up to three fatty acid residues, corresponding to the three alcoholic groups on glycerin.

In some embodiments, the unsaturated glycerides are triglycerides, which contain three fatty acid residues, which can be the same or different. In some such embodiments, the triglycerides contain one unsaturated fatty acid residue and two saturated fatty acid residues. In some embodiments, the triglycerides contain two unsaturated fatty acid residues and one saturated fatty acid residue. In some embodiments, the triglycerides contain three unsaturated fatty acid residues.

In some embodiments, the nature of the triglyceride composition can depend on the source of the triglycerides. In some embodiments, the triglycerides are from a natural oil feedstock. In such embodiments, any suitable natural oil can be used. In some embodiments, the natural oil is rapeseed oil (canola oil), coconut oil, corn oil, cottonseed oil, olive oil, palm oil, peanut oil, safflower oil, sesame oil, soybean oil, sunflower oil, linseed oil, palm kernel oil, tung oil, jatropha oil, mustard seed oil, pennycress oil, camelina oil, hempseed oil, castor oil, or some combination thereof. In some embodiments, the natural oil is rapeseed oil (canola oil), palm oil, soybean oil, or some combination thereof.

In some embodiments, certain glycerides can contain two or three unsaturated fatty acid residues. In such embodiments, the glycerides not only can react with compounds of formula (II) via metathesis, but can also react with other unsaturated glycerides to form metathesis dimers, trimers, tetramers, pentamers, and the like of the glycerides. Thus, in some embodiments, the compound resulting from the reaction comprises a metathesis dimer of unsaturated glycerides. In some embodiments, the compound comprises a metathesis trimer of unsaturated glycerides. In some embodiments, the compound comprises a metathesis tetramer of unsaturated glycerides. In some embodiments, the compound comprises a metathesis pentamer of unsaturated glycerides. In some embodiments, the compound comprises a metathesis hexamer of unsaturated glycerides.

In some embodiments of any of the aforementioned embodiments, the one or more unsaturated glycerides comprise at least one saturated fatty acid residue. In some such embodiments, the at least one saturated fatty acid residue is selected independently from the group consisting of lauric acid residues, myristic acid residues, palmitic acid residues, and stearic acid residues.

In some embodiments of any of the aforementioned embodiments, the UV-stabilizing residue, for example, can be any suitable moiety that can improve the useful lifetime of a UV chemical filter. In some such embodiments, the UV-stabilizing residue can also be a moiety that absorbs UV radiation, such as UV-A and/or UV-B radiation. For example, in some embodiments, the UV-stabilizing residue is a derivative the UV-chemical filter. With reference to this context, the term “derivative” refers to moieties that have undergone simple transformations to facilitate their linkage as part of the compound of formula (II). Thus, in some embodiments, the UV-stabilizing residue is a derivative of a UV chemical filter, such as a chemical filter that absorbs UV-A and/or UV-B radiation.

The UV-stabilizing residue can have any acceptable molecular weight. Thus, in some embodiments, the UV-stabilizing residue is a polymer. In some other embodiments, the UV-stabilizing residue is a small molecule, such as a moiety having a molecular weight of no more than 2000 Da, or no more than 1500 Da, or no more than 1000 Da, or no more than 750 Da, or no more than 500 Da.

In some embodiments, the UV-stabilizing residue operates by accepting a photon from the UV-chemical filter. Thus, the UV-stabilizing residue absorbs UV radiation at approximately the same wavelength as the UV chemical filter that is being stabilized. Therefore, some UV-stabilizing residues can stabilize UV-A radiation, UV-B radiation, or both UV-A and UV-B radiation, e.g., by being able to absorb a photon having a wavelength in that range(s) of UV-A and/or UV-B radiation. In some embodiments, the UV-stabilizing residue is a moiety that stabilizes chemical filters of UV-A radiation, e.g., by absorbing a photon of UV-A radiation from the chemical filter. In some embodiments, the UV-stabilizing residue is a moiety that stabilizes chemical filters of UV-B radiation, e.g., by absorbing a photon of UV-B radiation from the chemical filter.

For compounds of formula (II), R²¹ can contain any suitable UV-stabilizing moiety. In some embodiments, the UV-stabilizing residue is a dinaphthalate, a salicylate, a crylene, a fluorine, a camphor, a syringlidene malonate, a polysilicone, a polyester, a benzotriazole, a triazine, a methoxycinnamate, a sulfonic acid, a benzone, or a benzoic acid.

In some embodiments, the compound of formula (II) is:

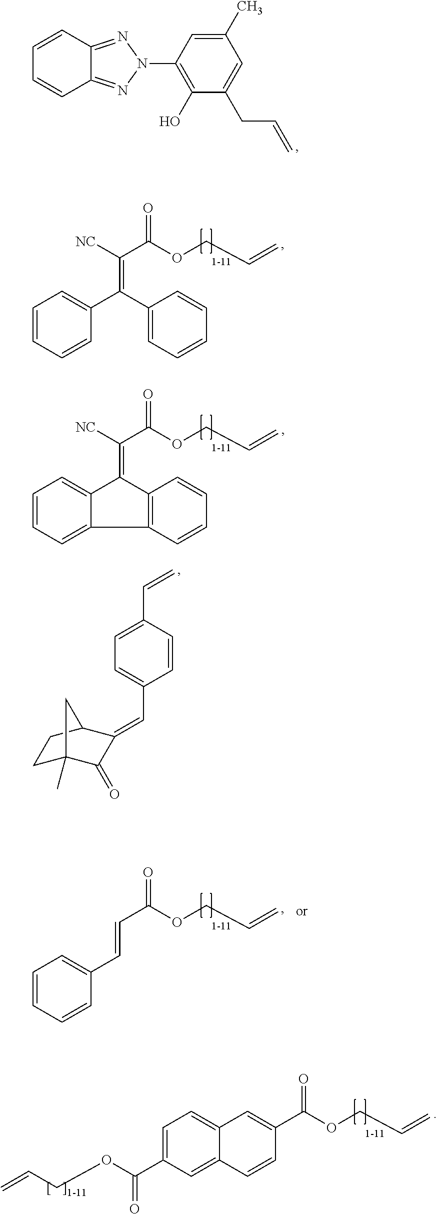

In some embodiments, the compounds formed according any of the aforementioned embodiments can be partially or fully hydrogenated using standard hydrogenation techniques known in the art.

### Methods for Making UV-Stabilizing Compounds

In further aspects, the disclosure provides methods for making a photostabilizing composition, the method comprising: providing (a) one or more unsaturated glycerides, and (b) one or more olefinically functionalized UV-stabilizing compounds; and reacting the one or more unsaturated glycerides and the one or more olefinically functionalized UV-stabilizing compounds in the presence of a metathesis catalyst.

Any suitable unsaturated glycerides can be used. In some embodiments, the unsaturated glycerides comprise at least one unsaturated fatty acid residue, such as, for example, oleic acid residues, a linoleic acid residues, and/or a linolenic acid residue. Such glycerides can have up to three fatty acid residues, corresponding to the three alcoholic groups on glycerin.

In some embodiments, the unsaturated glycerides are triglycerides, which contain three fatty acid residues, which can be the same or different. In some such embodiments, the triglycerides contain one unsaturated fatty acid residue and two saturated fatty acid residues. In some embodiments, the triglycerides contain two unsaturated fatty acid residues and one saturated fatty acid residue. In some embodiments, the triglycerides contain three unsaturated fatty acid residues.

In some embodiments, the nature of the triglyceride composition can depend on the source of the triglycerides. In some embodiments, the triglycerides are from a natural oil feedstock. In such embodiments, any suitable natural oil can be used. In some embodiments, the natural oil is rapeseed oil (canola oil), coconut oil, corn oil, cottonseed oil, olive oil, palm oil, peanut oil, safflower oil, sesame oil, soybean oil, sunflower oil, linseed oil, palm kernel oil, tung oil, jatropha oil, mustard seed oil, pennycress oil, camelina oil, hempseed oil, castor oil, or some combination thereof. In some embodiments, the natural oil is rapeseed oil (canola oil), palm oil, soybean oil, or some combination thereof.

In some embodiments, certain glycerides can contain two or three unsaturated fatty acid residues. In such embodiments, the glycerides not only can react with compounds of formula (II) via metathesis, but can also react with other unsaturated glycerides to form metathesis dimers, trimers, tetramers, pentamers, and the like of the glycerides. Thus, in some embodiments, the compound resulting from the reaction comprises a metathesis dimer of unsaturated glycerides. In some embodiments, the compound comprises a metathesis trimer of unsaturated glycerides. In some embodiments, the compound comprises a metathesis tetramer of unsaturated glycerides. In some embodiments, the compound comprises a metathesis pentamer of unsaturated glycerides. In some embodiments, the compound comprises a metathesis hexamer of unsaturated glycerides.

In some embodiments of any of the aforementioned embodiments, the one or more unsaturated glycerides comprise at least one saturated fatty acid residue. In some such embodiments, the at least one saturated fatty acid residue is selected independently from the group consisting of lauric acid residues, myristic acid residues, palmitic acid residues, and stearic acid residues.

In some embodiments of any of the aforementioned embodiments, the one or more olefinically functionalized UV-stabilizing compounds are compounds formed by reacting a UV-stabilizing compound with an olefin, wherein the olefin comprises a functional group having chemical reactivity toward one or more sites on the UV-stabilizing compound.

In some such embodiments, the UV-stabilizing compound has a molecular weight of no more than 2000 Da, or no more than 1500 Da, or no more than 1000 Da, or no more than 750 Da, or no more than 500 Da.

In some such embodiments, the UV-stabilizing compound stabilizes chemical filters of UV-A radiation. In some such embodiments, the UV-stabilizing compound stabilizes chemical filters of UV-B radiation. In some such embodiments, the UV-stabilizing compound is a compound that stabilizes chemical filters of UV-A radiation and UV-B radiation.

In some embodiments, the UV-stabilizing compound is a dinaphthalate, a salicylate, a crylene, a fluorine, a camphor, a syringlidene malonate, a polysilicone, a polyester, a benzotriazole, a triazine, a methoxycinnamate, a sulfonic acid, a benzone, or a benzoic acid. For example, in some embodiments, the UV-stabilizing compound is undecylcrylene dimethicone, butyloctyl salicylate, ethylhexyl methoxycrylene, dimethyl capramide, methylbenzylidene camphor, diethylhexyl 2,6-naphthalate, benzylidene dimethoxydimethylindanone, diethylhexyl syringylidene malonate, bis-ethylhexyl hydroxydimethoxy benzylmalonate, methylene bis-benzotriazolyl tetramethylbutylphenol, bis-ethylhexyloxyphenol methoxyphenyl triazine, diethylamino hydroxybenzoyl hexyl benzoate, tris-biphenyl triazine, octocrylene, terephthalylidene dicamphor sulfonic acid, 2-hydroxy sulfobetaine of a cinnamidoalkyl amine, amino-substituted hydroxybenzophenones, or (2-hydroxy-4-methoxyphenyl)-phenylmethanone.

In some embodiments of any of the aforementioned embodiments, the one or more olefinically functionalized UV-stabilizing compounds are selected from:

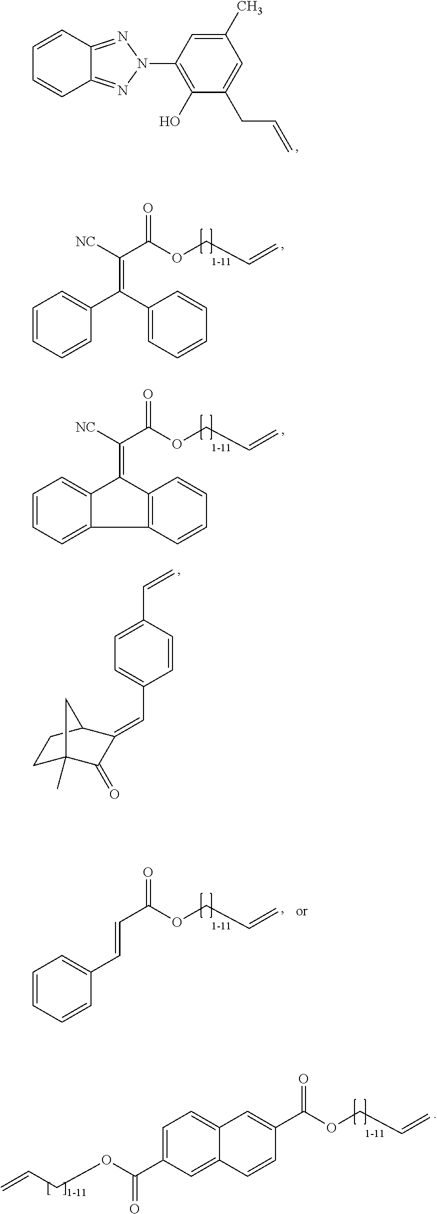

Personal Care and/or Sunscreen Compositions

In further aspects, the disclosure provides compositions that comprise one or more compounds of any of the aforementioned aspects and/or embodiments. In some embodiments, the compositions are personal care compositions, such as a face cream, a skin lotion, and the like. In some embodiments, the compositions are sunscreen compositions, such as spreadable sunscreen compositions or sprayable sunscreen compositions.

The composition can be in any suitable form, such as a single-phase composition, or as a multiple-phase emulsion. In some embodiments, the composition is an emulsion having a dispersed phase and a continuous phase, wherein the dispersed phase comprises one or more compounds of any of the aforementioned aspects and/or embodiments, and wherein the continuous phase comprises water. In some other embodiments, the composition is an emulsion having a dispersed phase and a continuous phase, wherein the continuous phase comprises one or more compounds of any of the aforementioned aspects and/or embodiments, and wherein the dispersed phase comprises water.

The composition can contain any suitable amount of the compounds of formula (I) or compounds made by the aforementioned reaction of compounds of formula (II). In some embodiments, the composition comprises the compounds in amounts of 0.1 percent by weight up to 5 percent by weight, or up to 10 percent by weight, or up to 15 percent by weight, or up to 20 percent by weight.

Further, in some embodiments, the composition can contain one or more additional ingredients. In some embodiments, these ingredients are selected from the group consisting of: topical carriers, emollients, light-scattering particles, UV-absorbing compounds, other UV-stabilizing compounds, film-forming polymers, surfactants, pigments, and fragrances.

Derivation from Renewable Sources

The compounds employed in any of the aspects or embodiments disclosed herein can, in certain embodiments, be derived from renewable sources, such as from various natural oils or their derivatives. Any suitable methods can be used to make these compounds from such renewable sources. Suitable methods include, but are not limited to, fermentation, conversion by bioorganisms, and conversion by metathesis.

Olefin metathesis provides one possible means to convert certain natural oil feedstocks into olefins and esters that can be used in a variety of applications, or that can be further modified chemically and used in a variety of applications. In some embodiments, a composition (or components of a composition) may be formed from a renewable feedstock, such as a renewable feedstock formed through metathesis reactions of natural oils and/or their fatty acid or fatty ester derivatives. When compounds containing a carbon-carbon double bond undergo metathesis reactions in the presence of a metathesis catalyst, some or all of the original carbon-carbon double bonds are broken, and new carbon-carbon double bonds are formed. The products of such metathesis reactions include carbon-carbon double bonds in different locations, which can provide unsaturated organic compounds having useful chemical properties.

A wide range of natural oils, or derivatives thereof, can be used in such metathesis reactions. Examples of suitable natural oils include, but are not limited to, vegetable oils, algae oils, fish oils, animal fats, tall oils, derivatives of these oils, combinations of any of these oils, and the like. Representative non-limiting examples of vegetable oils include rapeseed oil (canola oil), coconut oil, corn oil, cottonseed oil, olive oil, palm oil, peanut oil, safflower oil, sesame oil, soybean oil, sunflower oil, linseed oil, palm kernel oil, tung oil, jatropha oil, mustard seed oil, pennycress oil, camelina oil, hempseed oil, and castor oil. Representative non-limiting examples of animal fats include lard, tallow, poultry fat, yellow grease, and fish oil. Tall oils are by-products of wood pulp manufacture. In some embodiments, the natural oil or natural oil feedstock comprises one or more unsaturated glycerides (e.g., unsaturated triglycerides). In some such embodiments, the natural oil feedstock comprises at least 50% by weight, or at least 60% by weight, or at least 70% by weight, or at least 80% by weight, or at least 90% by weight, or at least 95% by weight, or at least 97% by weight, or at least 99% by weight of one or more unsaturated triglycerides, based on the total weight of the natural oil feedstock.

The natural oil may include canola or soybean oil, such as refined, bleached and deodorized soybean oil (i.e., RBD soybean oil). Soybean oil typically includes about 95 percent by weight (wt %) or greater (e.g., 99 wt % or greater) triglycerides of fatty acids. Major fatty acids in the polyol esters of soybean oil include but are not limited to saturated fatty acids such as palm itic acid (hexadecanoic acid) and stearic acid (octadecanoic acid), and unsaturated fatty acids such as oleic acid (9-octadecenoic acid), linoleic acid (9,12-octadecadienoic acid), and linolenic acid (9,12,15-octadecatrienoic acid).

Metathesized natural oils can also be used. Examples of metathesized natural oils include but are not limited to a metathesized vegetable oil, a metathesized algal oil, a metathesized animal fat, a metathesized tall oil, a metathesized derivatives of these oils, or mixtures thereof. For example, a metathesized vegetable oil may include metathesized canola oil, metathesized rapeseed oil, metathesized coconut oil, metathesized corn oil, metathesized cottonseed oil, metathesized olive oil, metathesized palm oil, metathesized peanut oil, metathesized safflower oil, metathesized sesame oil, metathesized soybean oil, metathesized sunflower oil, metathesized linseed oil, metathesized palm kernel oil, metathesized tung oil, metathesized jatropha oil, metathesized mustard oil, metathesized camelina oil, metathesized pennycress oil, metathesized castor oil, metathesized derivatives of these oils, or mixtures thereof. In another example, the metathesized natural oil may include a metathesized animal fat, such as metathesized lard, metathesized tallow, metathesized poultry fat, metathesized fish oil, metathesized derivatives of these oils, or mixtures thereof.

Such natural oils, or derivatives thereof, can contain esters, such as triglycerides, of various unsaturated fatty acids. The identity and concentration of such fatty acids varies depending on the oil source, and, in some cases, on the variety. In some embodiments, the natural oil comprises one or more esters of oleic acid, linoleic acid, linolenic acid, or any combination thereof. When such fatty acid esters are metathesized, new compounds are formed. For example, in embodiments where the metathesis uses certain short-chain olefins, e.g., ethylene, propylene, or 1-butene, and where the natural oil includes esters of oleic acid, an amount of 1-decene and 1-decenoid acid (or an ester thereof), among other products, are formed. Following transesterification, for example, with an alkyl alcohol, an amount of 9-denenoic acid alkyl ester is formed. In some such embodiments, a separation step may occur between the metathesis and the transesterification, where the alkenes are separated from the esters. In some other embodiments, transesterification can occur before metathesis, and the metathesis is performed on the transesterified product.

In some embodiments, the natural oil can be subjected to various pre-treatment processes, which can facilitate their utility for use in certain metathesis reactions. Useful pre-treatment methods are described in United States Patent Application Publication Nos. 2011/0113679, 2014/0275595, and 2014/0275681, all three of which are hereby incorporated by reference as though fully set forth herein.

In some embodiments, after any optional pre-treatment of the natural oil feedstock, the natural oil feedstock is reacted in the presence of a metathesis catalyst in a metathesis reactor. In some other embodiments, an unsaturated ester (e.g., an unsaturated glyceride, such as an unsaturated triglyceride) is reacted in the presence of a metathesis catalyst in a metathesis reactor. These unsaturated esters may be a component of a natural oil feedstock, or may be derived from other sources, e.g., from esters generated in earlier-performed metathesis reactions. In certain embodiments, in the presence of a metathesis catalyst, the natural oil or unsaturated ester can undergo a self-metathesis reaction with itself.

In some embodiments, the metathesis comprises reacting a natural oil feedstock (or another unsaturated ester) in the presence of a metathesis catalyst. In some such embodiments, the metathesis comprises reacting one or more unsaturated glycerides (e.g., unsaturated triglycerides) in the natural oil feedstock in the presence of a metathesis catalyst. In some embodiments, the unsaturated glyceride comprises one or more esters of oleic acid, linoleic acid, linoleic acid, or combinations thereof. In some other embodiments, the unsaturated glyceride is the product of the partial hydrogenation and/or the metathesis of another unsaturated glyceride (as described above).

The conditions for such metathesis reactions, and the reactor design, and suitable catalysts are as described below with reference to the metathesis of the olefin esters. That discussion is incorporated by reference as though fully set forth herein.

### Olefin Metathesis

In some embodiments, one or more of the unsaturated monomers can be made by metathesizing a natural oil or natural oil derivative. The terms “metathesis” or “metathesizing” can refer to a variety of different reactions, including, but not limited to, cross-metathesis, self-metathesis, ring-opening metathesis, ring-opening metathesis polymerizations (“ROMP”), ring-closing metathesis (“RCM”), and acyclic diene metathesis (“ADMET”). Any suitable metathesis reaction can be used, depending on the desired product or product mixture.

In some embodiments, after any optional pre-treatment of the natural oil feedstock, the natural oil feedstock is reacted in the presence of a metathesis catalyst in a metathesis reactor. In some other embodiments, an unsaturated ester (e.g., an unsaturated glyceride, such as an unsaturated triglyceride) is reacted in the presence of a metathesis catalyst in a metathesis reactor. These unsaturated esters may be a component of a natural oil feedstock, or may be derived from other sources, e.g., from esters generated in earlier-performed metathesis reactions. In certain embodiments, in the presence of a metathesis catalyst, the natural oil or unsaturated ester can undergo a self-metathesis reaction with itself.

In some embodiments, the metathesis comprises reacting a natural oil feedstock (or another unsaturated ester) in the presence of a metathesis catalyst. In some such embodiments, the metathesis comprises reacting one or more unsaturated glycerides (e.g., unsaturated triglycerides) in the natural oil feedstock in the presence of a metathesis catalyst. In some embodiments, the unsaturated glyceride comprises one or more esters of oleic acid, linoleic acid, linoleic acid, or combinations thereof. In some other embodiments, the unsaturated glyceride is the product of the partial hydrogenation and/or the metathesis of another unsaturated glyceride (as described above).

The metathesis process can be conducted under any conditions adequate to produce the desired metathesis products. For example, stoichiometry, atmosphere, solvent, temperature, and pressure can be selected by one skilled in the art to produce a desired product and to minimize undesirable byproducts. In some embodiments, the metathesis process may be conducted under an inert atmosphere. Similarly, in embodiments where a reagent is supplied as a gas, an inert gaseous diluent can be used in the gas stream. In such embodiments, the inert atmosphere or inert gaseous diluent typically is an inert gas, meaning that the gas does not interact with the metathesis catalyst to impede catalysis to a substantial degree. For example, non-limiting examples of inert gases include helium, neon, argon, and nitrogen, used individually or in with each other and other inert gases.

The rector design for the metathesis reaction can vary depending on a variety of factors, including, but not limited to, the scale of the reaction, the reaction conditions (heat, pressure, etc.), the identity of the catalyst, the identity of the materials being reacted in the reactor, and the nature of the feedstock being employed. Suitable reactors can be designed by those of skill in the art, depending on the relevant factors, and incorporated into a refining process such, such as those disclosed herein.

The metathesis reactions disclosed herein generally occur in the presence of one or more metathesis catalysts. Such methods can employ any suitable metathesis catalyst. The metathesis catalyst in this reaction may include any catalyst or catalyst system that catalyzes a metathesis reaction. Any known metathesis catalyst may be used, alone or in combination with one or more additional catalysts. Examples of metathesis catalysts and process conditions are described in US 2011/0160472, incorporated by reference herein in its entirety, except that in the event of any inconsistent disclosure or definition from the present specification, the disclosure or definition herein shall be deemed to prevail. A number of the metathesis catalysts described in US 2011/0160472 are presently available from Materia, Inc. (Pasadena, Calif.).

In some embodiments, the metathesis catalyst includes a Grubbs-type olefin metathesis catalyst and/or an entity derived therefrom. In some embodiments, the metathesis catalyst includes a first-generation Grubbs-type olefin metathesis catalyst and/or an entity derived therefrom. In some embodiments, the metathesis catalyst includes a second-generation Grubbs-type olefin metathesis catalyst and/or an entity derived therefrom. In some embodiments, the metathesis catalyst includes a first-generation Hoveyda-Grubbs-type olefin metathesis catalyst and/or an entity derived therefrom. In some embodiments, the metathesis catalyst includes a second-generation Hoveyda-Grubbs-type olefin metathesis catalyst and/or an entity derived therefrom. In some embodiments, the metathesis catalyst includes one or a plurality of the ruthenium carbene metathesis catalysts sold by Materia, Inc. of Pasadena, Calif. and/or one or more entities derived from such catalysts. Representative metathesis catalysts from Materia, Inc. for use in accordance with the present teachings include but are not limited to those sold under the following product numbers as well as combinations thereof: product no. C823 (CAS no. 172222-30-9), product no. C848 (CAS no. 246047-72-3), product no. C601 (CAS no. 203714-71-0), product no. C627 (CAS no. 301224-40-8), product no. C571 (CAS no. 927429-61-6), product no. C598 (CAS no. 802912-44-3), product no. C793 (CAS no. 927429-60-5), product no. C801 (CAS no. 194659-03-9), product no. C827 (CAS no. 253688-91-4), product no. C884 (CAS no. 900169-53-1), product no. C833 (CAS no. 1020085-61-3), product no. C859 (CAS no. 832146-68-6), product no. C711 (CAS no. 635679-24-2), product no. C933 (CAS no. 373640-75-6).

In some embodiments, the metathesis catalyst includes a molybdenum and/or tungsten carbene complex and/or an entity derived from such a complex. In some embodiments, the metathesis catalyst includes a Schrock-type olefin metathesis catalyst and/or an entity derived therefrom. In some embodiments, the metathesis catalyst includes a high-oxidation-state alkylidene complex of molybdenum and/or an entity derived therefrom. In some embodiments, the metathesis catalyst includes a high-oxidation-state alkylidene complex of tungsten and/or an entity derived therefrom. In some embodiments, the metathesis catalyst includes molybdenum (VI). In some embodiments, the metathesis catalyst includes tungsten (VI). In some embodiments, the metathesis catalyst includes a molybdenum- and/or a tungsten-containing alkylidene complex of a type described in one or more of (a) Angew. Chem. Int. Ed. Engl., 2003, 42, 4592-4633; (b) Chem. Rev., 2002, 102, 145-179; and/or (c) Chem. Rev., 2009, 109, 3211-3226, each of which is incorporated by reference herein in its entirety, except that in the event of any inconsistent disclosure or definition from the present specification, the disclosure or definition herein shall be deemed to prevail.

In certain embodiments, the metathesis catalyst is dissolved in a solvent prior to conducting the metathesis reaction. In certain such embodiments, the solvent chosen may be selected to be substantially inert with respect to the metathesis catalyst. For example, substantially inert solvents include, without limitation: aromatic hydrocarbons, such as benzene, toluene, xylenes, etc.; halogenated aromatic hydrocarbons, such as chlorobenzene and dichlorobenzene; aliphatic solvents, including pentane, hexane, heptane, cyclohexane, etc.; and chlorinated alkanes, such as dichloromethane, chloroform, dichloroethane, etc. In some embodiments, the solvent comprises toluene.

In other embodiments, the metathesis catalyst is not dissolved in a solvent prior to conducting the metathesis reaction. The catalyst, instead, for example, can be slurried with the natural oil or unsaturated ester, where the natural oil or unsaturated ester is in a liquid state. Under these conditions, it is possible to eliminate the solvent (e.g., toluene) from the process and eliminate downstream olefin losses when separating the solvent. In other embodiments, the metathesis catalyst may be added in solid state form (and not slurried) to the natural oil or unsaturated ester (e.g., as an auger feed).

The metathesis reaction temperature may, in some instances, be a rate-controlling variable where the temperature is selected to provide a desired product at an acceptable rate. In certain embodiments, the metathesis reaction temperature is greater than −40° C., or greater than −20° C., or greater than 0° C., or greater than 10° C. In certain embodiments, the metathesis reaction temperature is less than 200° C., or less than 150° C., or less than 120° C. In some embodiments, the metathesis reaction temperature is between 0° C. and 150° C., or is between 10° C. and 120° C.

### EXAMPLES

### Example 1

### Self-Metathesized Canola Oil (Comparative Example)

Thermally pre-treated and degassed canola oil (207 g, 0.929 mol olefin equivalent as determined by iodine value titration) was added to a dry 500-mL multi-neck round-bottom flask. The reactor was equipped with an internal thermocouple, shortpath distillation head, receiving flask, stirbar, and resistive heating mantle. The reactor and contents were inerted via nitrogen sparging for 30 minutes, after which time the dip tube was raised from beneath the oil surface. The reactor was warmed to 65° C. Olefin metathesis catalyst C827 (8.28 mg, 207 μL of a 40.0 mg/mL solution in anhydrous, oxygen-free toluene) (Materia, Inc., Pasadena, Calif., USA) was introduced via syringe. The ensuing metathesis reaction was digested for 2-3 hours at 65° C., with stirring, until equilibrium product concentrations had been achieved. The reaction was cooled to ambient temperature, then ethyl vinyl ether (200 μL) was added beneath the product polyglyceride surface to deactive catalyst. Low boiling hydrocarbons were removed under reduced pressure (5-10 mmHg, 120° C.). The product, canola oil polyglyceride, was obtained as a viscous, clear amber oil. Analysis by gas chromatography indicated that the average molecular weight (peak average) of the product is 10648 g/mol. The resulting composition is referred to herein as Composition 1.

### Example 2A

### Self-Metathesized Canola Oil with 0.5% UV Stabilizer Derivative

Thermally pre-treated and degassed canola oil (200.37 g, 0.891 mol olefin equivalent as determined by iodine value titration) and 2-allyl-6-(2H-benzotriazol-2-yl)-p-cresol (1.198 g, 4.51 mmol) were added to a dry 500 mL multi-neck round bottom flask. The reactor was equipped with an internal thermocouple, shortpath distillation head, receiving flask, stirbar, and resistive heating mantle. The reactor and contents were inerted via nitrogen sparging for 30 minutes, after which time the dip tube was raised from beneath the oil surface. The reactor was warmed to 65° C. Olefin metathesis catalyst C827 (8.42 mg, 200.0 μL of a 42.1 mg/mL C827 solution in anhydrous, oxygen-free toluene) was introduced via syringe. The ensuing metathesis reaction was digested for 2-3 hours at 65° C., with stirring, until equilibrium product concentrations had been achieved. The reaction was cooled to ambient temperature, then ethyl vinyl ether (200 μL) was added beneath the product polyglyceride surface to deactivate catalyst. Low boiling hydrocarbons were removed under reduced pressure (5-10 mmHg, 120° C.). The product, canola oil-photostabilizer polyglyceride adduct (188.14 g, 93.9% yield) was obtained as a viscous, clear amber oil. GC-MS analysis confirmed complete consumption of 2-allyl-6-(2H-benzotriazol-2-yl)-p-cresol and the formation of polymer-bound metathesis adducts.

NMR analysis of the resulting polyglyceride further supported the incorporation of the heterocyclic aromatic 2-allyl-6-(2H-benzotriazol-2-yl)-p-cresol moiety. ¹H NMR δ (ppm): 7.0 (1H, d), 7.42 (2H, m), 7.78 (2H, m), 8.03 (1H, d), 11.30 (1H, t). Analysis by gas chromatography confirmed the consumption of 2-allyl-6-(2H-benzotriazol-2-yl)-p-cresol, and indicated that the average molecular weight (peak average) of the product is 10414 g/mol. The resulting composition is referred to herein as Composition 2A.

### Example 2B

### Self-Metathesized Canola Oil with 1.0% UV Stabilizer Derivative

Thermally pre-treated and degassed canola oil (199.94 g, 0.889 mol olefin equivalent as determined by iodine value titration) and 2-allyl-6-(2H-benzotriazol-2-yl)-p-cresol (2.3607 g, 8.89 mmol) were added to a dry 500 mL multi-neck round bottom flask. The reactor was equipped with an internal thermocouple, shortpath distillation head, receiving flask, stirbar and resistive heating mantle. The reactor and contents were inerted via nitrogen sparging for 30 minutes, after which time the dip tube was raised from beneath the oil surface. The reactor was warmed to 65° C. Olefin metathesis catalyst C827 (8.24 mg, 200.0 μL of a 41.2 mg/mL C827 solution in anhydrous, oxygen-free toluene) was introduced via syringe. The ensuing metathesis reaction was digested for 2-3 hours at 65° C., with stirring, until equilibrium product concentrations had been achieved. The reaction was cooled to ambient temperature, then ethyl vinyl ether (200 μL) was added beneath the product polyglyceride surface to deactivate catalyst. Low boiling hydrocarbons were removed under reduced pressure (5-10 mmHg, 120° C.). The product, canola oil polyglyceride, was obtained as a viscous, clear amber oil. GC-MS analysis confirmed complete consumption of 2-allyl-6-(2H-benzotriazol-2-yl)-p-cresol and the formation of polymer-bound metathesis adducts.

NMR analysis of the resulting polyglyceride further supported the incorporation of the heterocyclic aromatic 2-allyl-6-(2H-benzotriazol-2-yl)-p-cresol moiety. ¹H NMR δ (ppm): 7.0 (1H, d), 7.42 (2H, m), 7.78 (2H, m), 8.03 (1H, d), 11.30 (1H, t). Analysis by gas chromatography confirmed the consumption of 2-allyl-6-(2H-benzotriazol-2-yl)-p-cresol, and indicated that the average molecular weight (peak average) of the product is 10357 g/mol. The resulting composition is referred to herein as Composition 2B.

### Example 2C

### Self-Metathesized Canola Oil with 2.0% UV Stabilizer Derivative

Thermally pre-treated and degassed canola oil (202.49 g, 0.900 mol olefin equivalent as determined by iodine value titration) and 2-allyl-6-(2H-benzotriazol-2-yl)-p-cresol (4.78 g, 18.02 mmol) were added to a dry 500 mL multi-neck round bottom flask. The reactor was equipped with an internal thermocouple, shortpath distillation head, receiving flask, stirbar and resistive heating mantle. The reactor and contents were inerted via nitrogen sparging for 30 minutes, after which time the dip tube was raised from beneath the oil surface. The reactor was warmed to 65° C. Olefin metathesis catalyst C827 (8.00 mg, 200.0 μL of a 40.0 mg/mL C827 solution in anhydrous, oxygen-free toluene) was introduced via syringe. The ensuing metathesis reaction was digested for 2-3 hours at 65° C., with stirring, until equilibrium product concentrations had been achieved. The reaction was cooled to ambient temperature, then ethyl vinyl ether (200 μL) was added beneath the product polyglyceride surface to deactivate catalyst. Low boiling hydrocarbons were removed under reduced pressure (5-10 mmHg, 120° C.). The product, canola oil-photostabilizer polyglyceride adduct (197.96 g, 97.7% yield) was obtained as a viscous, clear amber oil. GC-MS analysis confirmed complete consumption of 2-allyl-6-(2H-benzotriazol-2-yl)-p-cresol and the formation of polymer-bound metathesis adducts.

NMR analysis of the resulting polyglyceride further supports the incorporation of the heterocyclic aromatic 2-allyl-6-(2H-benzotriazol-2-yl)-p-cresol moiety. ¹H NMR δ (ppm): 7.0 (1H, d), 7.42 (2H, m), 7.78 (2H, m), 8.03 (1H, d), 11.30 (1H, t). Analysis by gas chromatography confirmed the consumption of 2-allyl-6-(2H-benzotriazol-2-yl)-p-cresol, and indicated that the average molecular weight is 7948 g/mol. The resulting composition is referred to herein as Composition 2C.

### Example 3

### UV-Vis Analysis

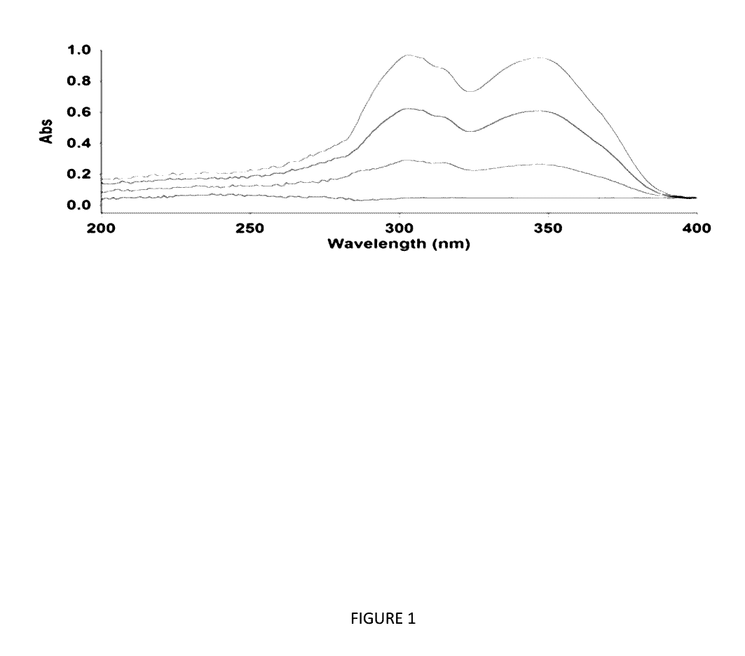

Compositions 1, 2A, 2B, and 2C were analyzed to determine their UV absorption as a function of wavelength. The resulting spectrum for each compound is shown in FIG. 1. From bottom to top, the four curves correspond to Compositions 1, 2A, 2B, and 2C, respectively. Thus, Composition 2C shows the highest degree of UV light absorption.

### Example 4

### Avobenzone Stability

Absorption of UV-A radiation prompts the tautomerization of avobenzone from its keto-enol form to its β-diketo form. Upon UV-B radiation absorption of the latter, avobenzone irreversibly degrades and is rendered ineffective. To ascertain the protective properties of the novel polyglycerides (i.e., Compositions 2A, 2B, and 2C), a controlled avobenzone UV exposure study was undertaken, in the absence and presence of photostabilizing material. In the study, production of the β-diketo form of avobenzone (λmax˜290 nm) was observed in the absence of a photostabilizing additive. In the presence of Composition 2C, under the same set of exposure conditions, no absorption increase at the 290 nm. This observation indicated that tautomerization of avobenzone to its degradation-susceptible form is mitigated by the presence of Composition 2C.

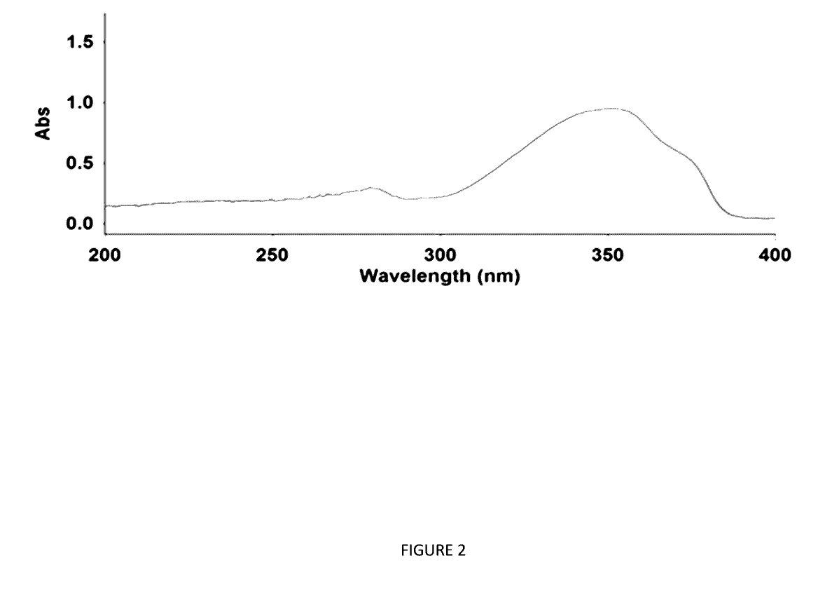

FIG. 2 shows the results of the experiment described above using Composition 1 (comparative) in combination with avobenzone. The lower curve shows the UV absorption prior to UV light exposure, while the upper curve shows the UV absorption after 45 minutes of UV light exposure. The peak that emerged at 290 nm corresponds to avobenzone degradation.

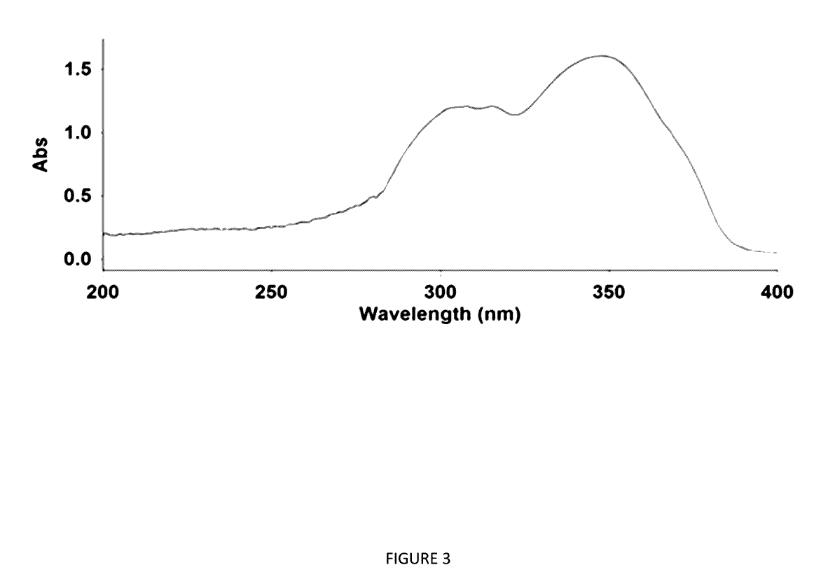

FIG. 3 shows the results of the experiment described above using Composition 2C in combination with avobenzone. Two curves are present in the figure, but they lie directly on top of each other, making it appear that only one curve is present. One curve shows the UV absorption prior to UV light exposure, while the other curve shows the UV absorption after 45 minutes of UV light exposure. The absence of any observable change indicates that effectively no avobenzone degraded upon 45 minutes of exposure to UV light.

1-62. (canceled)

63. A compound formed by reacting one or more unsaturated glycerides with a compound of formula (II)
HC═CH—R²¹ (II)
in the presence of a metathesis catalyst, wherein R²¹ is a moiety comprising a UV-stabilizing residue.

64. The compound of claim 63, wherein the one or more unsaturated glycerides comprise at least one unsaturated fatty acid residue.

65. The compound of claim 64, wherein the at least one unsaturated fatty acid residue is an oleic acid residue, a linoleic acid residue, or a linolenic acid residue.

66. The compound of claim 64, wherein at least one of the one or more unsaturated glycerides comprises two or three unsaturated fatty acid residues.

67. The compound of claim 66, wherein the two or three unsaturated fatty acid residues are selected independently from the group consisting of an oleic acid residue, a linoleic acid residue, and a linolenic acid residue.

68. The compound of claim 66, wherein the compound comprises a metathesis dimer of unsaturated glycerides.

69. The compound of claim 66, wherein the compound comprises a metathesis trimer of unsaturated glycerides.

70. The compound of claim 66, wherein the compound comprises a metathesis tetramer of unsaturated glycerides.

71. The compound of claim 66, wherein the compound comprises a metathesis pentamer of unsaturated glycerides.

72. The compound of claim 66, wherein the compound comprises a metathesis hexamer of unsaturated glycerides.

73. The compound of claim 63, wherein the UV-stabilizing residue is a dinaphthalate, a salicylate, a crylene, a fluorine, a camphor, a syringlidene malonate, a polysilicone, a polyester, a benzotriazole, a triazine, a methoxycinnamate, a sulfonic acid, a benzone, or a benzoic acid.

74. The compound of claim 63, wherein the one or more unsaturated glycerides are derived from a natural oil.

75. A composition comprising one or more compounds of claim 63.

76. The composition of claim 75, wherein the composition is an emulsion having a dispersed phase and a continuous phase, wherein the dispersed phase comprises the one or more compounds, and wherein the continuous phase comprises water.

77. The composition of claim 75, wherein the composition is an emulsion having a dispersed phase and a continuous phase, wherein the continuous phase comprises the one or more compounds, and wherein the dispersed phase comprises water.

78. The composition of claim 75, further comprising one or more ingredients selected from the group consisting of: topical carriers, emollients, light-scattering particles, UV-absorbing compounds, other UV-stabilizing compounds, film-forming polymers, surfactants, pigments, and fragrances.

79. A method for making a photostabilizing composition, the method comprising:
providing (a) one or more unsaturated glycerides, and (b) one or more olefinically functionalized UV-stabilizing compounds;
reacting the one or more unsaturated glycerides and the one or more olefinically functionalized UV-stabilizing compounds in the presence of a metathesis catalyst.

80. The method of claim 79, wherein the one or more unsaturated glycerides comprise at least one unsaturated fatty acid residue.

81. The method of claim 80, wherein the at least one unsaturated fatty acid residue is an oleic acid residue, a linoleic acid residue, or a linolenic acid residue.

82. The method of claim 79, wherein at least one of the one or more unsaturated glycerides comprises two or three unsaturated fatty acid residues.

83-92. (canceled)
